SEQUENCE LISTING (2 sequences)

SEQ ID NO: 1 - DNA, 437 bp, Homo sapiens
ggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 2 - DNA, 437 bp, Homo sapiens
ggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtgacaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

(19) United States
(12) Patent Application Publication
(10) Pub. No.: US 2004/0197315 A1
(43) Pub. Date: Oct. 7, 2004

(54) Dopaminergic cell lines stably expressing A53T alpha-synuclein and methods of using same

(76) Inventors: Leonidas Stefanis, Ilisia (GR); Lloyd A. Greene, Larchmont, NY (US)

Correspondence Address: Leslie Gladstone Restaino, 163 Madison Avenue, P.O. Box 1989, Morristown, NJ, 07962-1989, US

(21) Appl. No.: 10/737,262

(22) Filed: Dec. 15, 2003

Related U.S. Application Data
(60) Provisional application No. 60/433,450, filed on Dec. 13, 2002.

(51) Int. Cl. A61K 48/00; C12N 5/06; C12N 15/85

(52) U.S. Cl. 424/93.21; 435/455; 435/353

(57) ABSTRACT

The present invention discloses a PC12 cell line stably expressing human A53T α-synuclein, wherein a cell of the cell line is characterized by proteasomal dysfunction, dopaminergic dysfunction, lysosomal dysfunction, and increased non-apoptotic cell death, as compared to cells of suitable control cell line not expressing mutant α-synuclein. Also provided are methods of making the cells of the present invention. The unique phenotype associated with the cells described herein provide a cellular model for the study of Parkinson's Disease, as well as for other synucleinopathic neurodegenerative disorders, including, but not limited to, Dementia with Lewy Bodies, Lewy Body Variant of Alzheimer's Disease, Multiple System Atrophy and Hallervorden-Spatz syndrome.

### RELATED APPLICATION

This application claims the benefit of U.S. Provisional Application No. 60/433,450, filed Dec. 13, 2002.

### BACKGROUND OF THE INVENTION

Parkinson's disease (PD) is a neurodegenerative disorder characterized by resting tremor, rigidity, hypokinesia, and postural instability. Pathological examination shows loss of pigmented dopaminergic neurons and accumulation of eosinophilic inclusions termed Lewy bodies in the substantia nigra pars compacta and other brainstem nuclei (Fahn and Przedborski, Parkinsonism. In: Merritt's textbook of neurology, Ed 10 (Rowland L P, ed), pp 679-693. Philadelphia: Williams & Wilkins 2000). The cause of PD is unknown, but recent studies indicate that two separate α-synuclein mutations, A53T and A30P, are responsible for certain rare familial forms of the disease (Polymeropoulos et al., Mutation in the α-synuclein gene identified in families with Parkinson's disease. Science, 276:2045-2047, 1997; Kruger et al., Ala30Pro mutation in the gene encoding α-synuclein in Parkinson's disease. Nat. Genet., 18:106-108, 1998). α-Synuclein is a protein of unknown function that localizes within presynaptic terminals in the CNS (Clayton and George, The synucleins: a family of proteins involved in synaptic function, plasticity, neurodegeneration, and disease. Trends Neurosci., 21:249-254, 1998; Clayton and George, Synucleins in synaptic plasticity and neurodegenerative disorders. J. Neurosci. Res., 58:120-129, 1999) and, together with ubiquitin, is among the major components of Lewy bodies (Spillantini et al., α-Synuclein in Lewy bodies. Nature, 388:839-840, 1997; Spillantini, et al., α-Synuclein in filamentous inclusions of Lewy bodies from Parkinson's disease and dementia with Lewy bodies. Proc. Natl. Acad. Sci. USA, 95:6469-6473, 1998), suggestive again of an association with PD pathogenesis.

Two other genetic causes of PD have been identified. Mutations in the gene encoding Parkin are identified in cases with autosomal recessive PD (Kitada et al., Mutations in the parkin gene cause autosomal recessive juvenile parkinsonism. Nature, 392:605-608, 1998), and a mutation in the gene encoding for ubiquitin C-terminal hydrolase L-1 (UCHL-1) is associated with PD in one family (Leroy et al., The ubiquitin pathway in Parkinson's disease. Nature, 395:451-452, 1998). Both of these proteins are involved in the ubiquitin-dependent degradation of intracellular proteins (Ciechanover, The ubiquitin-proteasome pathway: on protein death and cell life. EMBO J, 17:7151-7160, 1998). Proteins that are degraded through this system are tagged with polyubiquitin chains through a series of enzymatic reactions and then degraded by the proteasome, a multicatalytic complex. Some ubiquitinated proteins may also be degraded by the lysosomal system (Ciechanover, The ubiquitin-proteasome pathway: on protein death and cell life. EMBO J, 17:7151-7160, 1998). Parkin is an E3 ligase, responsible for the attachment of ubiquitin to substrates such as CDCrel-1, Pael receptor, and, possibly, a glycosylated form of α-synuclein (Shimura et al., Familial Parkinson disease gene product, parkin, is a ubiquitin-protein ligase. Nat. Genet., 25:302-305, 2000; Zhang et al., Parkin functions as an E2-dependent ubiquitin-protein ligase and promotes the degradation of the synaptic vesicle-associated protein, CDCrel-1. Proc. Natl. Acad. Sci. USA, 97:13354-13359, 2000; Imai et al., An unfolded putative transmembrane polypeptide, which can lead to endoplasmic reticulum stress, is a substrate of Parkin. Cell, 105:891-902, 2001; Shimura et al., Ubiquitination of a new form of α-synuclein by parkin from human brain: implications for Parkinson's disease. Science, 293:263-269, 2001), and UCHL-1 is a member of the family of deubiquitinating enzymes, which remove polyubiquitin chains once the substrate has been attached to the proteasome (Ciechanover, The ubiquitin-proteasome pathway: on protein death and cell life. EMBO J, 17:7151-7160, 1998).

Induced expression of wild-type or mutant α-synuclein has been achieved in a number of cellular systems. The results have been variable, with some studies reporting no effects with overexpression alone (Ko et al., Sensitization of neuronal cells to oxidative stress with mutated human α-synuclein. J. Neurochem., 75:2546-2554, 2000), others reporting adverse effects with wild-type or mutant α-synuclein overexpression (Ostrerova et al., α-Synuclein shares physical and functional homology with 14-3-3 proteins. J. Neurosci., 19:5782-5791, 1999; Saha et al., Induction of neuronal death by α-synuclein. Eur. J. Neurosci., 12:3073-3077, 2000), and others reporting adverse effects only with mutant overexpression (Zhou et al., Overexpression of human α-synuclein causes dopamine neuron death in rat primary culture and immortalized mesencephalon-derived cells. Brain Res., 866:33-43, 2000; Lee et al., Effect of the overexpression of wild-type or mutant α-synuclein on cell susceptibility to insult. J. Neurochem., 76:998-1009, 2001). Many studies have also reported increased toxicity of wild-type α-synuclein overexpression and, more consistently, mutant α-synuclein overexpression in cells only after challenge with a variety of agents, particularly those that induce oxidative stress (Kanda et al., Enhanced vulnerability to oxidative stress by α-synuclein mutations and C-terminal truncation. Neuroscience, 97:279-284, 2000; Ko et al., Sensitization of neuronal cells to oxidative stress with mutated human α-Synuclein. J. Neurochem., 75:2546-2554, 2000; Ostrerova-Golts et al., The A53T α-synuclein mutation increases iron-dependent aggregation and toxicity. J. Neurosci., 20:6048-6054, 2000; Tabrizi et al., Expression of mutant α-synuclein causes increased susceptibility to dopamine toxicity. Hum. Mol. Genet., 9:2683-2689, 2000).

However, despite a number of cellular systems known in the art, there is currently no adequate cellular model for the study of PD and other synucleinopathic neurodegenerative disorders associated with the expression of mutant α-synuclein. Specifically, there is no cellular model based on dopaminergic cells which stably express mutant α-synuclein, where the phenotype of those cells shows certain characteristics which are commonly associated with PD and related synucleinopathic neurodegenerative disorders, especially characteristics such as impaired cellular degradation (i.e., lysosomal and proteasomal dysfunction) and dopaminergic dysfunction.

Many of the cell lines stably expressing mutant α-synuclein are based upon non-dopaminergic cells, such as human derived embryonic kidney (HEK) cells. (see, e.g., Tabrizi et al., Expression of mutant α-synuclein causes increased susceptibility to dopamine toxicity. Hum. Mol. Genet., 9:2683-2689, 2000 and Lehmensiek, et al., Expression of mutant α-synucleins enhances dopamine transporter-mediated MPP+toxicity in vitro. NeuroReport, 13(19):1279-1283, 2002). In Lehmensiek, et al. (“Lehmensiek”), human embryonic kidney cell lines were established which stably co-express a mutant α-synuclein isoform and the human dopamine transporter. (Lehmensiek, et al., NeuroReport, 13(19):1279-1283, 2002, supra). Using this cell model, Lehmensiak investigated the effects of the mutant α-synucleins on the dopamine transporter mediated toxicity of the selective dopaminergic neurotoxin 1-methyl-4-phrnylpyridinium ion (rotenone) in vitro. In Tabrizi, et al., stable, inducible HEK cell lines expressing mutant α-synuclein were created to investigate the morphological and biochemical consequences of wild-type and mutant protein expression. (Tabrizi, Hum. Mol. Genet., 9:2683-2689, 2000, supra). However, neither of these cell lines is capable of directly investigating the effects of mutant α-synuclein expression on the dopaminergic system, since the host cells are not capable of synthesizing dopamine or of generating an evoked dopamine release. This is especially important since the dopaminergic phenotype is affected out of proportion to the degree of dopaminergic cell loss in PD.

Other cell lines known in the art express mutant α-synuclein in dopaminergic cell lines, but the expression is transient. Further, there have been no reports of dopaminergic system dysfunction in any of these cell lines. By way of example, Zhou, et al. (“Zhou”) investigated the effects of adenovirus-mediated α-synuclein overexpression on dopamine neurons in rat primary mesencephalic cultures and in a rat dopaminergic cell line—the large T-antigen immortalized, mesencephalon-derived 1RB3AN27. (Brain Res., 866(1-2):33-43, 2000). While Zhou found that the transient overexpression of mutant human α-synuclein in their cultures caused apoptotic cell death in both of the rat primary mesencephalic cells and in the 1RB3AN27 cells, and that mutant α-synuclein overexpression in these cell cultures increased the sensitivity of dopamine neurons to certain neurotoxins (i.e., 6-hydroxydopamine), no dysfunction of the dopaminergic pathway was noted. Further, transiently transfected cell cultures do not provide an optimal tool for creating a cellular model of PD, or for the testing of therapeutic agents that inhibit dysfunction associated with PD, since the transfected cells may be heterogeneous and non-uniform within each culture, they cannot be maintained indefinitely to study the effects of long term α-synuclein overexpression, and cannot, generally speaking, be produced in large amounts.

Finally, Tanaka, et al. (“Tanaka”) teach the generation of stable, inducible PC12 cell lines in which wild-type or mutant A30P α-synuclein can be derepressed using doxycycline. (Human Mol. Genet., 10(9): 919-926, 2001). The cell lines of Tanaka show decreased proteasomal activity and increased sensitivity to apoptotic cell death when treated with sub-toxic concentrations of an exogenous proteasome inhibitor. Significantly, however, Tanaka did not report any dysfunction of the dopaminergic pathway associated with A30P α-synuclein expression in PC12 cells, such as might be exemplified by the absence of dense core granules or the absence of an evoked dopamine release. A phenotype expressing a dopaminergic deficit would be most beneficial in creating a cellular model of PD. Further, Tanaka teaches away from the use of A53T α-synuclein in PC12 lines, stating both that (i) human A53T mutant α-synuclein may be better tolerated in rat cells than the A30P α-synuclein mutation, and (ii) data from α-synuclein transgenic flies indicate that flies expressing A30P show a significantly greater phenotype than those expressing A53T mutant m-synuclein.

Accordingly, in light of the state of the art known to the inventors, there exists a need for a well-characterized dopaminergic cell line, such as PC12, that stably expresses A53T mutant α-synuclein. Such a stably expressing cell line would preferably exhibit a phenotype characteristic of dopaminergic dysfunction, since dopaminergic deficit is closely associated with the symptomatology of PD and related synucleinopathic neurodegenerative disorders.

### SUMMARY OF THE INVENTION

The present invention discloses a method for generating a dopaminergic cell line stably expressing human A53T α-synuclein, the method comprising the steps of introducing an expression vector into a population of rat pheochromocytoma PC12 cells, wherein the expression vector comprises a sequence encoding human A53T α-synuclein operatively linked to and under the control of a promoter, and isolating those PC12 cell lines stably expressing human A53T α-synuclein. In a preferred method of the present invention, the cells of the isolated PC12 cell lines stably expressing human A53T α-synuclein exhibit (i) proteasomal dysfunction, (ii) dopaminergic dysfunction, (iii) lysosomal dysfunction and (iv) increased non-apoptotic cell death, each when compared to a cell from a suitable control cell line not expressing mutant α-synuclein.

Also disclosed herein is a method for identifying an agent that inhibits cellular degeneration associated with the expression of mutant α-synuclein. In this method, a PC12 cell line stably expressing human A53T α-synuclein is obtained, wherein the cells of the cell line are characterized by increased non-apoptotic cell death. The cells of the PC12 cell line are contacted with an agent of interest, so that the phenotype of the contacted cells may be compared with the phenotype of cells from a suitable control line (i.e., a cell line expressing human A53T α-synuclein, but not contacted with the agent of interest). Accordingly, the effect of the agent of interest on the contacted cells can then be determined, wherein a phenotype of the contacted cells associated with reduced cellular degeneration indicates that the agent of interest has an inhibitory effect on cellular degeneration associated with the expression of mutant α-synuclein.

In another embodiment of the present invention, a method of identifying an agent that inhibits dopaminergic dysfunction associated with the expression of mutant α-synuclein is disclosed. Cells from a PC12 cell line stably expressing human A53T α-synuclein are contacted with an agent of interest, wherein the cells of the cell line are characterized by dopaminergic dysfunction. The phenotype of the contacted cells is then compared with the phenotype of cells from a suitable control line, so that the effect of the agent of interest on the contacted cells may be determined. A phenotype of the contacted cells associated with increased dopaminergic function indicates that the agent of interest has an inhibitory effect on dopaminergic dysfunction associated with the expression of mutant α-Synuclein.

In yet another embodiment of the present invention, a method of identifying an agent that inhibits proteasomal dysfunction associated with the expression of mutant α-synuclein is disclosed, the method comprising the steps of: (a) obtaining a PC12 cell line stably expressing human A53T α-synuclein, wherein cells of the cell line are characterized by proteasomal dysfunction; (b) contacting the cells of the PC12 cell line with an agent of interest; (c) comparing the phenotype of the contacted cells with the phenotype of cells from a suitable control line; and (d) determining the effect of the agent of interest on the contacted cells, wherein a phenotype of the contacted cells associated with increased proteasomal activity indicates that the agent of interest has an inhibitory effect on proteasomal dysfunction associated with the expression of mutant α-synuclein.

Still further, a method of identifying an agent that inhibits lysosomal dysfunction associated with the expression of mutant alpha-synuclein is disclosed. In this method, a PC12 cell line stably expressing human A53T α-synuclein is obtained, wherein the cells of the cell line are characterized by lysosomal dysfunction. These cells are then contacted with an agent of interest, so that the phenotype of the contacted cells can be compared with the phenotype of cells from a suitable control line, and a potential effect of the agent of interest determined. A phenotype of the contacted cells associated with increased lysosomal activity indicates that the agent of interest has an inhibitory effect on lysosomal dysfunction associated with the expression of mutant α-synuclein.

Also disclosed herein is an agent identified by any of the methods of the present invention, where the agent is found to have an inhibitory effect on the cellular degeneration, proteasomal dysfunction, dopaminergic dysfunction, or lysosomal dysfunction associated with the expression of mutant α-synuclein.

Finally, a method is disclosed of screening an agent to determine its potential effectiveness in the treatment of a synucleinopathic neurodegenerative disorder, the method comprising the steps of: (a) obtaining a PC12 cell line stably expressing human A53T α-Synuclein, wherein cells of the cell line are characterized by: (i) proteasomal dysfunction; (ii) dopaminergic dysfunction; (iii) lysosomal dysfunction; and (iv) increased non-apoptotic cell death; (b) contacting the cells of the PC12 cell line with an agent of interest; (c) comparing the phenotype of the contacted cells with the phenotype of cells from a suitable control line; and (d) determining the effect of the agent of interest on the contacted cells. A phenotype of the contacted cells associated with (i) decreased proteasomal dysfunction; (ii) decreased dopaminergic dysfunction; (iii) decreased lysosomal dysfunction; or (iv) decreased non-apoptotic cell death, when compared to the cells of a suitable control line, indicates that the agent of interest is potentially effective in treating a synucleinopathic neurodegenerative disorder. The synucleinopathic neurodegenerative disorder, while preferably Parkinson's Disease, may be any one of a number of synucleinopathic neurodegenerative disorders associated with aberrant α-synuclein expression or function, including, but not limited to, Dementia with Lewy Bodies, Lewy Body Variant of Alzheimer's Disease, Multiple System Atrophy and Hallervorden-Spatz syndrome. The present invention also provides for the agent identified as being potentially effective in treating such a synucleinopathic neurodegenerative disorder.

Additional aspects of the present invention will be apparent in view of the description that follows.

### BRIEF DESCRIPTION OF THE FIGURES

The application file associated with the subject patent application contains at least one drawing executed in color. A copy of this patent application, or of any patent issuing therefrom, with color drawings will be provided by the United States Patent and Trademark Office upon request and payment of the necessary fee.

FIG. 1 illustrates various levels of expression of wild-type and A53T mutant α-synuclein in clonal PC12 cell lines. Various clonal PC12 cell lines were generated expressing empty vector (P1 and P2), wild-type α-synuclein (S4, S10, and S12), or A53T mutant α-synuclein (M1, M13, M15, and M18). Triton-soluble lysates (200 μg of protein) were generated, resolved by 13% SDS-PAGE, and immunoblotted with a monoclonal synuclein antibody (1:1000; Transduction Laboratories, Division of BD Biosciences, Mississauga, ON). Two separate blots are presented. In the blot on the left, a control lysate from rat cortex (30 μg) was used in the first lane. The asterisk indicates the broad 18 kDa band seen in the control lysate and in the lines expressing α-synuclein. Note the 45 kDa band (arrow) present in the control lysate, the empty-vector controls, and the overexpressing lines. In the blot on the right, lysates of the M15 and M18 lines were similarly generated and processed. Three more empty vector lines (P3, P4, and P5) were generated and showed a pattern that was similar to that seen for P1 and P2 on immunoblotting (Stefanis et al., Synuclein 1 is selectively upregulated in response to NGF treatment in PC12 cells. J. Neurochem., 76:1165-1176, 2001).

FIG. 2 depicts the morphological alterations in PC12 cells expressing A53T α-synuclein. Panel A depicts four photomicrographs of naive PC12 cells expressing empty vector (P1), wild-type α-synuclein (S12), or A53T mutant α-synuclein (M1 and M15), respectively. The asterisks indicate granular degenerating cells. The single arrowhead indicates very large cells. The double arrowhead indicates a neuritic-like extension in an M1 cell and a stellate appearance in a M15 cell. Panel B depicts photomicrographs of PC12 cells expressing wild-type α-synuclein (S12) or A53T mutant α-synuclein (M1) and treated for 9 d with NGF. A neuritic network is clearly present in the S12 cultures but not in the M1 cultures. Large number of degenerating cells are seen in the cultures derived from the M1 line.

FIG. 3 is a pictograph illustrating increased nonapoptotic death in PC12 cells expressing A53T α-synuclein. Panel A measures Trypan blue uptake in PC12 cells from various clonal lines. The percentage of Trypan blue-positive cells is reported as the mean±SEM (n=3). M1 and M15 lines (p<0.001, determined by one-way ANOVA with Neuman-Keuls post hoc tests) and, to a lesser extent, M13 (p<0.05) showed a higher percentage of Trypan-blue-positive cells compared with empty vector or wild-type synuclein overexpressors. In Panel B, PC12 cells from various clonal lines were fixed and stained with Hoechst 33342 and nuclear morphology was evaluated. The percentage of apoptotic nuclei for each line is reported as the mean±SEM (n=3). As a positive control, cells from the P1 line were deprived of serum overnight and subsequently fixed and stained with the Hoechst dye (SD).

FIG. 4 illustrates ubiquitinated aggregates in PC12 cells expressing A53T α-synuclein. In Panel A, PC12 cells from the various lines were fixed and stained with a monoclonal synuclein antibody (1:50; Transduction Laboratories, a division of BD Biosciences, Mississauga, Ontario) (left column; Syn), a polyclonal ubiquitin antibody (1:100; DakoCytomation, Denmark) (middle column; Ubi), and the Hoechst dye 33342 (1 μg/ml; Sigma-Aldrich, St. Louis, Mo.) (right column; Hoechst). Ubiquitin staining in control and wild-type α-synuclein expressors was diffuse and low level and was not evident with this exposure. In contrast, with identical exposure time, note the strong staining for ubiquitin in the cytoplasm of M1 and M15 cells. In the last row, a degenerating (deg) M1 cell that has lost nuclear staining shows even more discrete punctate ubiquitin staining. In Panel B, Confocal microscopy of M1 or M15 cells shows ubiquitin (left column), synuclein (middle column), and combined (right column) immunostaining. The intense, punctate nature of ubiquitin immunostaining is apparent. There is little synuclein immunoreactivity within these aggregates. Nuclear synuclein immunostaining is evident in the cell shown from the M1 line.

FIG. 5 illustrates immunoblots of electrophoresis gels that indicate an increase of multi-ubiquitinated proteins in Triton-insoluble lysates of PC12 cells expressing A53T α-synuclein. In Panel A, cell lysates (200 μg of protein) from various clonal lines were resolved on an 8% SDS-PAGE gel and immunoblotted with an anti-ubiquitin polyclonal antibody (1:1000; DakoCytomation, Denmark). The arrows indicate a doublet at 52-54 kDa that was selectively increased in M1 and M15 cell lysates. This blot represents one of two independent experiments, which yielded similar results. In Panel B, Triton-insoluble, sample buffer-soluble lysates (200 μg of protein) were resolved on an 8% SDS-PAGE gel and immunoblotted with an anti-ubiquitin polyclonal antibody (1:1000; DakoCytomation, Denmark). The arrows indicate bands at 52-54 that were more prominent in the M1 and M15 lines. The bracket indicates higher molecular mass proteins, which were more prominent in the M1 and M15 lines. The arrowhead indicates the end of the stacking gel. The bottom panel is from a longer exposure (3 min vs 1 min) of the same blot, at the level of 30-35 kDa. The bands seen are presumably background bands, with similar intensity across the lanes. Equal protein loading was also verified by Ponceau S staining. This blot represents one of three independent experiments, which yielded similar results, except for the fact that an increase in polyubiquitinated proteins was inconsistently found for M13. In Panel C, Triton-insoluble, sample buffer-soluble lysates (200 μg of protein) from various clonal lines were resolved on a 12% SDS-PAGE gel and immunoblotted with anti-synuclein. The arrow indicates the 18 kDa α-synuclein band. Note that the exposure for this blot was at least 10 times longer than for the blots used to obtain similar band intensities from Triton-soluble lysates. The blot was intentionally overexposed to detect low abundance α-synuclein-specific bands in the upper portion of the blot. Such bands were not seen. The bands detected at the top portion of the blot are presumably nonspecific background bands, because they are also seen in the empty-vector control lysates. The bracket indicates background bands at 30-35 kDa that are of similar intensity across the various samples. To ensure equal protein loading, the same samples were loaded on another gel and immunoblotted with an anti-actin monoclonal antibody (1:5000; Sigma-Aldrich, St. Louis, Mo.) (bottom panel). The arrow in the bottom panel indicates the 44 kDa actin band.

FIG. 6 is a pictograph illustrating a decrease in proteasomal activity in PC12 cell lines expressing mutant A53T α-synuclein. PC12 cell lysates were generated and assayed for chymotrypsin-like proteasomal activity. The results for each line are reported as mean±SEM (n=8 for S12 and M15; n=10 for P5 and M1). Both M1 and M15 showed a statistically significant decrease in proteasomal activity compared with S12 or P5 (Student's nonpaired t test; *p<0.05; **p<0.01).

FIG. 7 depicts electromicrographs illustrating features of autophagy in the A53T α-synuclein-expressing lines. Cells from the P1 (a), S12 (b), M1 (c), and M15 (d) clonal lines were processed for EM. Scale bar: a, 1 μm (applied to (a)-(d)). Note the normal appearance of the P1 and S12 cells, with only rare lysosomal-vacuolar structures (arrows in (a) and (b)). Dense core granules are indicated by arrowheads, and were actually more prominent overall in the S12 cells compared with the P1 cells. Such granules were entirely absent from the mutant lines. Note the granular-vacuolar appearance of cells from the mutant lines in (c) and d and the dense packaging with lysosomal-like structures that range from electron dense (large arrows) to vacuolar (arrowheads). On many occasions double membrane-bound structures (small arrows in (c) and (d)) were noted to engulf intracellular organelles, and in particular mitochondria. M1 (e) and M15 (f) cells are shown at a higher magnification. Scale bar: e, 0.5 μm. Note again the numerous membrane-bound structures. Some of these are reminiscent of multivesicular bodies (arrowhead in (f)). The small arrow in (f) shows one such structure within a vacuole. Note a degenerating mitochondrion (large arrow in (f)) and a double-membrane structure engulfing a mitochondrion (small arrow in (e)). The large arrow in (e) denotes a relatively electron-dense lysosomal-like structure with a double membrane.

FIG. 8 shows pictographs illustrating the absence of depolarization-induced dopamine release from PC12 cells expressing A53T α-synuclein. Cells from the P4, S12 (S12a and S12b, from two separate subclones in two different experiments), M1, M15, and M18 lines were plated on 24-well dishes, and depolarization-induced dopamine release (A) and intracellular dopamine (B) were assessed as described in the Examples below Reduced presence of acidic organelles in the A53T α-synuclein-expressing lines. A, Cells from the various lines were labeled with the dye Lysotracker red (50 nM; Molecular Probes) and then visualized under a fluorescent microscope with a 40× objective. Representative pictures are shown. Note the very low level of labeling in lines M1 and M15. The arrows denote a rare vacuolar/inclusion-like structure intensely labeled with Lysotracker. Similar results were achieved in five independent experiments. B, For superior resolution, cells labeled as in A were visualized with an oil-immersion 60× objective. Note the discrete punctate labeling of P1 and S12 and the absence of such labeling in M1 and M15. An example of a large accumulation of Lysotracker labeling is shown again for M15. The results are reported as mean±SD (n=4).

FIG. 9 depicts reduced presence of acidic organelles in the A53T α-synuclein-expressing lines. In Panel A, cells from the various lines were labeled with the dye Lysotracker red (50 nM; Molecular Probes, Oregon) and then visualized under a fluorescent microscope with a 40× objective. Representative pictures are shown. There is a very low level of labeling in lines M1 and M15. The arrows denote a rare vacuolar/inclusion-like structure intensely labeled with Lysotracker red. Similar results were achieved in five independent experiments. In Panel B, cells labeled as in A were visualized with an oil-immersion 60× objective, yielding greater resolution. There is discrete punctate labeling of P1 and S12 and the absence of such labeling in M1 and M15. An example of a large accumulation of Lysotracker labeling is shown again for M15.

FIG. 10 depicts reduced degradation of a lysosomal substrate in PC12 cells expressing A53T α-synuclein. PC12 cells from the various cell lines were incubated with 2 mg/ml Lysosensor Yellow/Blue Dextran for 20 hr, rinsed three times in complete medium, and then visualized under a 100× oil-immersion objective. Images were captured in a UV (top) or a rhodamine (bottom) filter. There is increased fluorescent signal in cells from the M1 and M15 lines in the UV range, indicative of accumulation of the substrate in nonacidic organelles, and the absence of fluorescence in the rhodamine spectrum, indicative of the absence of degradation of the substrate in acidic organelles. Identical exposure times were used across the various lines. The experiment was repeated twice with similar results.

FIG. 11 shows limited colocalization between α-synuclein and cathepsin D. PC12 cells from the S12 or M18 lines were fixed and immunostained with synuclein-1 and cathepsin D antibodies. Immunostaining was then evaluated by confocal microscopy. Representative pictures are shown. Limited colocalization was noted between cathepsin D (left column) and α-synuclein (middle column), and the extent of colocalization did not differ among the two cell lines. Combined images are shown in the right column. A similar immunostaining pattern was seen in M1 and M15 cells (data not shown).

FIG. 12 illustrates a suggested model of cellular dysfunction and death induced by mutant α-synuclein.

### DETAILED DESCRIPTION OF THE INVENTION

To investigate the effects of wild-type or mutant α-synuclein in a dopaminergic cell system, the inventors have generated stable rat pheochromocytoma PC12 cell lines (Greene and Tischler, Establishment of a noradrenergic clonal line of rat adrenal pheochromocytoma cells which respond to nerve growth factor. Proc. Natl. Acad. Sci. USA, 73:2424-2428, 1976) expressing the wild-type and A53T mutant forms of the human protein.

Particular emphasis has been placed on the effects of α-synuclein on the cellular degradation machinery, because of the genetic and pathological evidence suggesting dysfunction of this system in PD (Kitada et al., Mutations in the parkin gene cause autosomal recessive juvenile parkinsonism. Nature, 392:605-608, 1998; Leroy et al., The ubiquitin pathway in Parkinson's disease. Nature 395:451-452, 1998; Shimura et al., Familial Parkinson disease gene product, parkin, is a ubiquitin-protein ligase. Nat. Genet., 25:302-305, 2000; McNaught and Jenner, Proteasomal function is impaired in substantia nigra in Parkinson's disease. Neurosci. Lett., 297:191-194, 2001; McNaught et al., Failure of the ubiquitin-proteasome system in Parkinson's disease. Nat. Rev. Neurosci., 2:589-594, 2001), and on the dopaminergic phenotype, because this seems to be affected out of proportion to the degree of dopaminergic cell loss in PD (Hornykiewicz, Dopamine (3-hydroxytyramine) and brain function. Pharmacol. Rev., 18:925-965, 1996). Expression of mutant α-synuclein in the cell lines of the present invention: (i) enhances baseline levels of death, (ii) induces accumulation of autophagic-vesicular structures, and (iii) results in a loss of catecholamine storage granules, and consequently, the capacity for depolarization-induced dopamine release. These effects are accompanied by, and may be consequences of, defects in the lysosomal and proteasomal degradation systems.

Accordingly, and in light of the foregoing, a method is disclosed for generating dopaminergic cell lines stably expressing human wild type and human A53T α-Synuclein. As used herein, a “dopaminergic” cell line refers to a cell line constituted by cells capable of synthesizing and releasing the neurotransmitter dopamine. In general, these cells contain a chemical pathway that changes the amino acid tyrosine into L-dopa, and then into dopamine. While the dopaminergic cells of the present invention may constitute any cell line capable of synthesizing and releasing dopamine (i.e., dopamine neurons derived from mesencephalic cultures, mesencephalon-derived 1RB3AN27 cells, SH-SY5Y cells, etc.), in a preferred embodiment of the invention, the dopaminergic cells are PC12 cells derived from rat adrenal pheochromocytoma (Greene, et al., Establishment of a noradrenergic clonal line of rat adrenal pheochromocytoma cells which respond to nerve growth factor. Proc. Natl. Acad. Sci. USA, 73(7):2424-8, 1976; Tischler, et al., Morphological and cytochemical properties of a clonal line of rat adrenal pheochromocytoma cells which respond to nerve growth factor. Lab. Invest., 39(2):77-89, 1978; ATCC Accession No. CRL-1721). PC12 cells are preferred not only because they are dopaminergic, but also because they have been extensively studied as models of neuronal degeneration (Greene, Nerve growth factor prevents the death and stimulates the neuronal differentiation of clonal PC12 pheochromocytoma cells in serum-free medium. J. Cell. Biol., 78:747-755, 1978; Batistatou and Greene, Aurintricarboxylic acid rescues PC12 cells and sympathetic neurons from cell death caused by nerve growth factor deprivation: correlation with suppression of endonuclease activity. J. Cell. Biol., 115:461-471, 1991; Rukenstein et al., Multiple agents rescue PC12 cells from serum-free cell death by translation- and transcription-independent mechanisms. J. Neurosci., 11:2552-2563, 1991; Mesner et al., Nerve growth factor withdrawal-induced cell death in neuronal PC12 cells resembles that in sympathetic neurons. J. Cell. Biol., 119:1669-1680, 1992; Stefanis et al., Induction of CPP32-like activity in PC12 cells by withdrawal of trophic support. J. Biol. Chem., 271:30663-30671, 1996).

Accordingly, in a preferred embodiment, the method of the present invention comprises the step of introducing an expression vector into a population of rat pheochromocytoma PC12 cells, wherein the expression vector comprises a sequence encoding human A53T α-synuclein operatively linked to and under the control of a promoter. PC12 cell lines stably expressing the human A53T α-synuclein are then isolated to generate separate clonal lines. As used herein, the human A53T α-synuclein is “stably expressed” when the expression vector comprising the human A53T α-synuclein sequence is inherited, in integrated or episomal form, from one generation to the next within an isolated clonal cell line.

Also disclosed herein is a method whereby the expression vector introduced into a population of rat pheochromocytoma PC12 cells comprises a sequence encoding human wild-type α-synuclein operatively linked to and under the control of a promoter, so that PC12 cell lines stably expressing human wild-type α-synuclein may be isolated. In a preferred embodiment of the invention, the sequence encoding human A53T α-synuclein comprises the nucleic acid sequence of SEQ ID NO.:2, while the sequence encoding human wild-type α-synuclein preferably comprises the nucleic acid sequence of SEQ ID NO.: 1.

As used herein, the term “expression vector” refers to a vector or vehicle comprising a nucleic acid sequence coding for at least part of a gene product (i.e., human wild-type or A53T α-synuclein), wherein the nucleic acid sequence coding for the gene product is operably linked to and under the control of various regulatory sequences, such as promoters, attenuators, enhancers, polyadenylation signals, terminators, and the like, that provide for the expression of the coding sequence in a host cell. Any one of a wide number of expression vectors comprising the regulatory sequences necessary for the transcription and translation of wild-type or A53T α-synuclein in PC12 cells may be used, including, but not limited to, plasmid vectors, phasmid vectors, adenoviral vectors, adenoassociated vectors, vaccinia viral vectors, lentiviral vectors, herpes viral vectors, or retroviral vectors.

In the illustrated embodiment, the expression vector is the plasmid vector pcDNA3 (Invitrogen, Carlsbad, Calif.). However, other suitable expression vectors are well known in the art, and may include pET-3d (Novagen, Madison, Wis.), pProEx-1 (Invitrogen, Life Technologies), pFastBac 1 (Invitrogen, Life Technologies), pSFV (Invitrogen, Life Technologies), pcDNA II (Invitrogen), pSL301 (Invitrogen), pSE280 (Invitrogen), pSE380 (Invitrogen), pSE420 (Invitrogen), pTrcHis A,B,C (Invitrogen), pRSET A,B,C (Invitrogen), pYES2 (Invitrogen), pAC360 (Invitrogen), pVL1392 and pV11392 (Invitrogen), pCDM8 (Invitrogen), pcDNA I (Invitrogen), pcDNA I(amp) (Invitrogen), pZeoSV (Invitrogen), pRc/CMV (Invitrogen), pRc/RSV (Invitrogen), pREP4 (Invitrogen), pREP7 (Invitrogen), pREP8 (Invitrogen), pREP9 (Invitrogen), pREP10 (Invitrogen), pCEP4 (Invitrogen), pCI (Promega), pSI (Promega) and pEBVHis (Invitrogen).

A typical expression vector used in the present invention would comprise eukaryotic DNA elements that control initiation of transcription, such as a promoter, as well as DNA elements that control the processing of transcripts, such as a transcription termination/polyadenylation sequence. Suitable promoters would be strong constitutive promoters, including, but not limited to, the adenovirus major late promoter, the promoter of the mouse metallothionein I gene, the TK promoter of Herpes virus, the SV40 early promoter, the Rous sarcoma virus promoter, the cytomegalovirus promoter, and the mouse mammary tumor virus promoter. Alternatively, the promoter may be an inducible promoter, whereby the transcriptional initiation activity of the promoter can be modified by alteration of conditions external to the cell, usually by the addition of a non-toxic molecule or a change in physical culture conditions. Examples of suitable inducible promoters include, but are not limited to, a tetracycline-responsive promoter, an ecdysone-inducible promoter, a metallothionein-regulated promoter, a steroid-regulated promoter, and a heat-shock regulated promoter. In an additional embodiment, a Tet-Off or Tet-On expression system may be used (Clontech, a Division of BD Biosciences, Palo Alto, Calif.). In the Tet-off system, gene expression is turned on when tetracycline or doxycycline is removed from the culture medium. In the “Tet-On” system, gene expression is activated by the addition of doxycycline.

The expression vector of the present invention may be introduced into the cells of a PC12 cell line by any suitable means, including, where appropriate, viral mediated transfection, or alternatively, such methods as calcium phosphate transfection, liposome-mediated transfection, microprojectile-mediated delivery, electroporation, biolistic transfection, or microinjection. In a preferred embodiment, a non-viral expression vector is introduced via electroporation or liposomal mediated transfection, as these methods minimize insult to the transfected cell and thereby maximize cell survival.

Following introduction of the expression vector, clonal PC12 cell lines stably expressing human A53T or wild-type α-synuclein must be isolated. In a preferred method of the present invention, the expression vector comprises a sequence encoding a selectable marker, so that those cells stably expressing human A53T or wild-type α-synuclein may be isolated by means of growth on a selective medium. A wide variety of selectable marker genes are known in the art and may be used in the method of the present invention. Most common selectable marker genes used for eukaryotic expression vectors include: the neo gene from the bacterial transposon Tn5 (which encodes for aminoglycoside phosphotransferase and thereby allows for cell selection on media containing G418 or neomycin like antibiotics); the zeo gene from the bacterial transposon Tn5 (which encodes for a bleomycin binding protein, and thereby allows for growth on media containing bleomycin or commercially available Zeo, an antibiotic that binds DNA and blocks RNA synthesis); the hyg gene from Eschericia coli (which encodes for hygromycin-B-transferase, and thereby allows for selection in media containing hygromycin-B, an aminocyclitol that inhibits protein synthesis); the gpt gene isolated from Eschericia coli (which encodes for xanthine-guanine-phosphoribosyltransferase, thereby allowing for growth in guanine-deficient media that contain inhibitors of de novo GMP synthesis and xanthine); and the pac gene from Streptomyces alboniger (which encodes for puromycin-N-acetyl transferase, and thereby allows for selection in media containing puromycin, an antibiotic that inhibits protein synthesis). Alternatively, markers that introduce an altered phenotype, such as green fluorescent protein from Aequorea victoria, or cell surface proteins such as CD4, CD8, Class 1 MHC, or placental alkaline phosphatase may be used to sort transfected cells from untransfected cells by such means as FACS sorting or magnetic bead separation technology. In a preferred embodiment, however, the expression vector comprises the selectable marker gene neo, thereby allowing for the selection of PC12 cells stably expressing human A53T α-Synuclein by virtue of growth on a selective medium comprising G-418 or neomycin.

The cells of the isolated cell lines preferably exhibit a unique phenotype characterized by one or all of (i) proteasomal dysfunction, (ii) lysosomal dysfunction, (iii) dopaminergic dysfunction and (iv) cellular degeneration, each as compared to a cell from a suitable control cell line, i.e., a PC12 cell line not expressing mutant α-synuclein. Proteasomal dysfunction may be determined in a number of ways, including, but not limited to, the increased presence of ubiquinated aggregates in the cytoplasm of a cell of the isolated cell line, and a significantly decreased proteasomal chymotrypsin-like activity in a cell of the isolated cell line. Lysosomal dysfunction may be determined by the accumulation of lysosomal-autophagic structures in a cell of the cell line, decreased punctate staining of the cytoplasm with a suitable ionic dye, and decreased acidification or degradation of a lysosomal substrate, such as lysosensor-tagged dextran, by a cell of the cell line. Dopaminergic dysfunction may be determined by an absence of dense core granules in a cell of the cell line, an absence of evoked dopamine release by a cell of the cell line, or a reduced level of dopamine in the cytoplasm of a cell of the cell line. Cellular degeneration may be determined by a increased rate of non-apoptotic cell death in cells of the isolated cell line.

Accordingly, the present invention further discloses a PC12 cell line stably expressing human A53T α-synuclein, wherein a cell of the cell line is characterized by: (i) proteasomal dysfunction; (ii) dopaminergic dysfunction; (iii) lysosomal dysfunction; and (iv) increased non-apoptotic cell death, each as compared to a cell from a suitable control cell line, i.e., a PC12 cell line not expressing mutant α-synuclein. The PC12 cell line of the present invention is preferably further characterized by: (i) the presence of ubiquinated aggregates in the cytoplasm; (ii) significantly decreased proteasomal chymotrypsin-like activity; (iii) an absence of dense core granules; (iv) an absence of evoked dopamine release; and (v) an accumulation of lysosomal-autophagic structures associated with non-apoptotic cell death. The noted characteristics of this preferred cell line render it well suited as a cellular model of Parkinson's disease.

The present invention further discloses a method of identifying an agent that inhibits a detrimental phenotype associated with mutant α-synuclein expression. In one embodiment, a method is disclosed of identifying an agent that inhibits the cellular degeneration associated with the expression of mutant α-synuclein. In this method, a PC12 cell line stably expressing human A53T α-synuclein is obtained, wherein the cells of the cell line are characterized by increased non-apoptotic cell death. These cells are then contacted with the agent of interest, which might be a drug, a compound, a nucleic acid, protein, peptide or antibody. The phenotype of the contacted cells is then compared with the phenotype of cells from a suitable control line, such as a cell line expressing human A53T α-synuclein that has not been exposed to the agent of interest. The effect of the agent of interest on the contacted cells can then be determined, wherein a phenotype of the contacted cells associated with reduced cellular degeneration (relative to the cells of the suitable control line) indicates that the agent of interest has an inhibitory effect on cellular degeneration associated with the expression of mutant α-synuclein. Specifically, a phenotype associated with reduced cellular degeneration includes a reduction of non-apoptotic cell death in the contacted cells as compared to cells of a suitable control line. Preferentially, the cells of the cell line stably expressing human A53T α-synuclein are further characterized by: (i) proteasomal dysfunction; (ii) dopaminergic dysfunction; and (iii) lysosomal dysfunction.

In an alternate embodiment, a method of identifying an agent that inhibits dopaminergic dysfunction associated with the expression of mutant α-synuclein is disclosed, the method comprising the steps of: (a) obtaining a PC12 cell line stably expressing human A53T α-synuclein, wherein cells of the cell line are characterized by dopaminergic dysfunction; (b) contacting the cells of the PC12 cell line with an agent of interest; (c) comparing the phenotype of the contacted cells with the phenotype of cells from a suitable control line; and (d) determining the effect of the agent of interest on the contacted cells, wherein a phenotype of the contacted cells associated with increased dopaminergic function (relative to the cells from a suitable control cell line) indicates that the agent of interest has an inhibitory effect on dopaminergic dysfunction associated with the expression of mutant α-synuclein. A phenotype associated with increased dopaminergic function may include an increase of intracellular dopamine levels, the ability of a contacted cell to undergo evoked dopamine release, or the increased presence of dense core granules in the cytoplasm of the contacted cells.

In yet another alternate embodiment, a method of identifying an agent that inhibits proteasomal dysfunction associated with the expression of mutant α-synuclein is disclosed. In this method, a PC12 cell line stably expressing human A53T α-synuclein is obtained, wherein the cells of the cell line are characterized by proteasomal dysfunction. The cells are then contacted with an agent of interest, and the phenotype of the contacted cells is compared with the phenotype of cells from a suitable control line. A phenotype of the contacted cells associated with increased proteasomal activity indicates that the agent of interest has an inhibitory effect on proteasomal dysfunction associated with the expression of mutant α-synuclein. Such a phenotype may be characterized in a number of ways, including, but not limited to, an observed increase of proteasomal chymotrypsin-like activity in the contacted cells, or an observed decrease in the presence of ubiquinated aggregates in the contacted cells.

Still further, in another alternate embodiment, a method of identifying an agent that inhibits lysosomal dysfunction associated with the expression of mutant alpha-synuclein is disclosed, the method comprising the steps of: (a) obtaining a PC12 cell line stably expressing human A53T α-synuclein, wherein cells of the cell line are characterized by lysosomal dysfunction; (b) contacting the cells of the PC12 cell line with an agent of interest; (c) comparing the phenotype of the contacted cells with the phenotype of cells from a suitable control line; and (d) determining the effect of the agent of interest on the contacted cells, wherein a phenotype of the contacted cells associated with increased lysosomal activity indicates that the agent of interest has an inhibitory effect on lysosomal dysfunction associated with the expression of mutant α-synuclein. Increased lysosomal activity may be indicated by a reduced presence of lysosomal-autophagic structures in the cytoplasm of the contacted cells, an increase of fine, punctate staining with an ionic dye in the contacted cells, or by an increase of acidification and degradation of a lysosomal substrate in the contacted cells.

Finally, a method of screening an agent to determine its potential effectiveness in the treatment of a synucleinopathic neurodegenerative disorder is disclosed, the method comprising the steps of: (a) obtaining a PC12 cell line stably expressing human A53T α-Synuclein, wherein cells of the cell line are characterized by: (i) proteasomal dysfunction; (ii) dopaminergic dysfunction; (iii) lysosomal dysfunction; and (iv) increased non-apoptotic cell death; (b) contacting the cells of the PC12 cell line with an agent of interest; (c) comparing the phenotype of the contacted cells with the phenotype of cells from a suitable control line; and (d) determining the effect of the agent of interest on the contacted cells, wherein a phenotype of the contacted cells associated with (i) decreased proteasomal dysfunction; (ii) decreased dopaminergic dysfunction; (iii) decreased lysosomal dysfunction; or (iv) decreased non-apoptotic cell death, when compared to the cells of a suitable control line, indicates that the agent of interest is potentially effective in treating a synucleinopathic neurodegenerative disorder. The synucleinopathic neurodegenerative disorder may be one of Parkinson's Disease, Dementia with Lewy Bodies, Lewy Body Variant of Alzheimer's Disease, Multiple System Atrophy (including Shy-Drager Syndrome, striatonigral degeneration, and olivopontocerebellar atrophy) and Hallervorden-Spatz syndrome, but is preferably Parkinson's Disease. The present invention also provides for the agents identified by the methods disclosed herein above.

The present invention is described in the following Examples, which are set forth to aid in the understanding of the invention, and should not be construed to limit in any way the scope of the invention as defined in the claims that follow thereafter.

### EXAMPLES

### Example 1

Cell Culture

PC12 cells (ATCC No. CRL-1721, ATCC, Manassas, Va.) were grown as described previously (Greene and Tischler, Establishment of a noradrenergic clonal line of rat adrenal pheochromocytoma cells which respond to nerve growth factor. Proc. Natl. Acad. Sci. USA, 73:2424-2428, 1976; Rukenstein et al., Multiple agents rescue PC12 cells from serum-free cell death by translation- and transcription-independent mechanisms. J. Neurosci., 11:2552-2563, 1991) on rat-tail collagen-coated dishes in RPMI 1640 medium, with 2 mM L-glutamine adjusted to contain 1.5 g/L sodium bicarbonate, 4.5 g/L glucose, 10 mM HEPES, 1.0 mM sodium pyruvate (to total 85%), 5% fetal bovine serum and 10% heat-inactivated horse serum (complete medium).

### Example 2

Generation of Constructs and Transfection of PC12 Cells

Wild-type α-synuclein in TA vector (Invitrogen, Carlsbad, Calif.) was generated as described previously (Stefanis et al., Synuclein 1 is selectively upregulated in response to NGF treatment in PC12 cells. J. Neurochem., 76:1165-1176, 2001). Specifically, RNA from post mortem material of human substantia nigra was extracted and reverse transcribed (RT, Roche Applied Science, Indianapolis, Ind.) into cDNA using previously published procedures well known in the art (see e.g., Neystat, et al., Alpha-synuclein expression in substantia nigra and cortex in Parkinson's disease. Mov. Disord., 14:417-422, 1999). The PCR was used to amplify the ORF of human α-synuclein, which was then cloned into a TA cloning vector. The insert was sequenced and was found to be identical to the reported sequence of NACP, also known as α-synuclein (Ueda, et al., Molecular cloning of cDNA encoding an unrecognized component of amyloid in Alzheimer's Disease. Proc. Natl. Acad. Sci. USA, 90:11282-11286, 1993; GenBank Accession No. NM007308 for transcript variant NACP 112; GenBank Accession No. NM000345 for transcript variant NACP 140). The A53T mutation was induced by PCR-based site-directed mutagenesis to yield the cDNA of human A53T mutant α-synuclein. Both inserts, constituting residues 33 to 469 of full length human α-synuclein (wild type α-synuclein insert is SEQ ID NO.: 1; A53T mutant α-synuclein is SEQ ID NO.: 2), were subcloned in the HindIII-XhoI sites of a PCDNA3 expression vector (Invitrogen, Carlsbad, Calif.), downstream of a cytomegalovirus promoter. PC12 cells were transfected by electroporation with empty vector, wild-type α-synuclein, or mutant α-Synuclein; individual colonies were subsequently selected in the presence of neomycin (Greene et al., Culture and experimental use of the PC12 rat pheochromocytoma cell line. In: Culturing nerve cells (Banker G, Goslin K, eds), pp 161-189. Cambridge, Mass.: MIT Press, 1998; Stefanis et al., 2001, supra).

### Example 3

Preparation of Cell Lysates for Western Blotting

Cells were rinsed in cold PBS and then collected in a buffer of 25 mM HEPES, pH 7.5, 5 mM EDTA, 1 mM EGTA, 5 mM MgCl₂, 0.5% Triton X-100, 2 mM DTT, 10 μg/ml each of pepstatin and leupeptin, and 1 mM PMSF. The cellular material was left for 20 min on ice. The lysate was then centrifuged for 20 min at 160,000×g, and the supernatant was collected. The detergent-insoluble pellet was solubilized in SDS-sample buffer and sonicated. Protein concentrations were measured using the Bradford assay (Bio-Rad, Richmond, Calif.).

### Example 4

Western Immunoblotting

Equal volumes of 2× sample buffer were added to Triton-soluble lysates (200 μg of protein). Alternatively, Triton-insoluble material solubilized in sample buffer (200 μg of protein) was used. The samples were boiled for 5 min, resolved by SDS-PAGE electrophoresis, transferred to nitrocellulose membranes, and immunoblotted with mouse anti-synuclein-1 (1:1000; Transduction Laboratories, Lexington, Ky.), rabbit anti-ubiquitin (1:1000; DakoCytomation, Glostrup, Denmark), or mouse anti-actin (1:5000, Sigma-Aldrich, St. Louis, Mo.) antibodies according to previously described procedures (Stefanis et al., Caspase-2 (Nedd2) processing and death of trophic factor-deprived PC12 cells and sympathetic neurons occur independently of caspase-3 (CPP32)-like activity. J. Neurosci., 18:9204-9215, 1998).

### Example 5

Assessment of Cell Death-Trypan Blue Assay and Nuclear Staining.

The day before the trypan blue assay, subconfluent cultures were rinsed twice with complete medium to remove cells at late stages of degeneration. The following day the cells were triturated off the dish and five drops of the cellular suspension were added in a tube together with five drops of Trypan blue solution (0.4%; Sigma-Aldrich, St. Louis, Mo.). After light mixing, the cells were placed on a glass slide, coverslipped, and visualized under 20× magnification. For each line, three fields of 100 cells each were assessed for the percentage of Trypan blue-positive cells. The results reported are the mean±SEM (n=3).

For nuclear staining, cells were plated in 35 mm collagen-coated dishes and then fixed and stained on the following day with the nuclear dye Hoechst 33342 (1 μg/ml; Sigma-Aldrich, St. Louis, Mo.).

### Example 6

Immunocytochemistry

PC12 cells from the various lines were fixed and immunostained with the mouse anti-synuclein-1 antibody (1:50) in combination with the rabbit anti-ubiquitin antibody (1:100) or a rabbit polyclonal antibody to cathepsin D (a generous gift from Dr. Eiki Kominami, Juntendo University, Tokyo, Japan; used at 1:200), using previously described procedures (Stefanis et al., 1999, supra; Stefanis, et al., 2001, supra). For indirect immunofluorescence studies, cells were plated on 35 mm dishes; for colocalization studies with confocal microscopy, cells were plated on glass coverslips coated with poly-D-lysine. For confocal microscopy, a Zeiss (Thornwood, N.Y.) LSM 410 scanning laser confocal attachment was used, mounted on a Zeiss Axiovert 100 TV inverted fluorescence microscope.

### Example 7

Assay for Chromostrypsin-like Activity of the Proteasome

Cells from the different lines were triturated off the dish, centrifuged, and washed in PBS. The resulting pellets were resuspended in 200-500 μl of lysis buffer (10 mM Tris-HCl, pH 7.8, with 1 mM ATP, and 10% glycerol) (Figueiredo-Pereira et al., A new inhibitor of the chymotrypsin-like activity of the multicatalytic proteinase complex (20S proteasome) induces accumulation of ubiquitin-protein conjugates in a neuronal cell. J. Neurochem., 63:1578-1581, 1994). The cells were left for 20 min on ice and then lysed with 30 strokes of a Dounce homogenizer. The lysates were then centrifuged at 10,000×g for 10 min. Fifty micrograms of protein of the resulting supernatants was included in an assay buffer of 100 mM Tris-HCl, pH 8.0, 2 mM CaCl₂, 1 mM ATP, and a 100 μM concentration of the fluorogenic substrate LLVY-7-amino-4-trifluoromethyl coumarin (AFC) (Enzyme Systems Products, Livermore, Calif.) in a total final volume of 1 ml. The assay was performed at 37° C. for 10 min (Figueiredo-Pereira et al., 1994, supra). The activity was then measured in a SLM 8000 fluorometer, with assay buffer without lysate as blank. The activity was linear with respect to the amount of protein (in the range of 25-200 μg of protein). In addition, lysates of cells treated overnight with the proteasomal inhibitor MG132 (0.5 μM; Biomol, Plymouth Meeting, Pa.) showed <5% of the activity, indicating that this activity was specific to the proteasome.

### Example 8

Electron Microscopy

Cells from the various lines were plated on poly-D-lysine and laminin-coated aclar in 35 mm dishes with punch holes. At 1-2 d after plating, the cells were rinsed with PBS and then fixed for 60 min at 4° C. with 2% glutaraldehyde in 2 mM CaCl₂ and 100 mM sodium cacodylate, pH 7.4. The fixed cultures were maintained in 100 mM sodium cacodylate and then processed for electron microscopy (EM) using standard methods (Tennyson et al., Structural abnormalities associated with congenital megacolon in transgenic mice that overexpress the Hoxa-4 gene. Dev. Dyn., 98:128-153, 1993).

### Example 9

Dopamine Release

Depolarization-induced dopamine release was quantified as described previously (Pothos et al., Synaptic vesicle transporter expression regulates vesicle phenotype and quantal size. J. Neurosci., 20:7297-7306, 2000) by HPLC coupled with electrochemical detection on an ESA (Bedford, Mass.) Coulochem II HPLC equipped with a model 5011 analytical cell with an applied potential of 400 mV and a Velosep RP-18 column (Applied Biosystems, Foster City, Calif.). The mobile phase contained 6.8 μm/l sodium acetate, 18.6 mg/l EDTA, 142 mg/l heptanesulfonic acid, and 10% methanol (adjusted to a pH of 4.6 with acetic acid). Briefly, cells from the various cell lines were plated in 24-well dishes. The cultures were rinsed once in PBS and then exposed to normal incubation medium (2 mM KCl) or to medium containing high potassium (80 mM KCl) for 1 min (Pothos et al., 2000, supra). The medium was then harvested in ice-cold 0.1N perchloric acid and analyzed for dopamine content. Depolarization-induced dopamine release was quantified as the difference in extracellular dopamine between cultures exposed to high potassium compared with normal incubation medium. To measure intracellular monoamine levels, medium was removed and the cells were rapidly solubilized in 100 μl of 0.3 M perchloric acid. The samples were centrifuged at 15,000 rpm for 15 min at 4° C. and stored at −80° C. until HPLC analysis.

### Example 10

Labeling of Living Cells and Labeling of Functional Lysosomes

Cells from the various lines were plated in 24-well dishes and incubated for 20 min at 37° C. with the cell-permeable dye Lysotracker red (50 nM; Molecular Probes, Eugene, Oreg.), which labels acidic organelles. Cells were then washed twice in PBS and visualized at 40× magnification under an epifluorescent microscope. In some experiments, for superior resolution, cells were plated on glass coverslips and visualized with oil immersion at 60× magnification in an inverted microscope.

To further investigate lysosomal function, cells were labeled with Lysosensor Yellow/Blue Dextran (Molecular Probes). This lysosensor-tagged dextran is endocytosed by the cells and degraded through the lysosomal system. It emits in the UV spectrum at a neutral pH and emits in the rhodamine-fluorescein spectrum at an acidic pH. Cells were plated from the different cell lines in 96 well plates and incubated the cultures with 2 mg/ml Lysosensor Yellow/Blue Dextran for various periods of time (8-20 hr). Cultures were rinsed three to four times with complete medium, plated on a glass slide, and covered with a glass coverslip. The cells were visualized with oil immersion at 100× magnification in an upright microscope. Images were obtained using the UV and the rhodamine filters. Representative pictures were obtained using identical exposure times across the different lines.

### Example 11

Results

A. Stable Expression of Wild-Type α-synuclein and A53T Mutant α-synuclein.

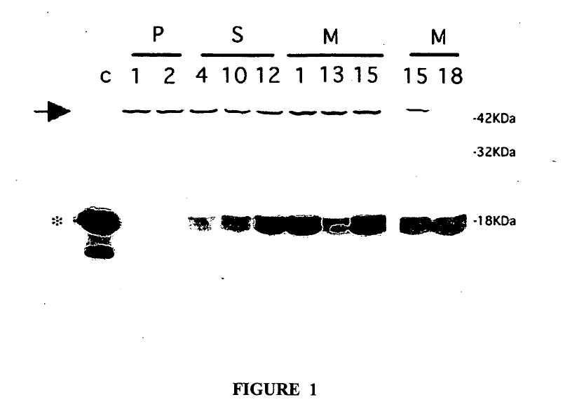

After the generation of various clonal lines transfected with empty vector, wild-type α-synuclein, and A53T mutant α-synuclein, Triton-soluble lysates were assessed by Western immunoblotting for the level of expression of α-synuclein. Only a small fraction of the detectable synuclein protein was Triton-insoluble. As reported previously (Stefanis et al., 2001, supra) in empty-vector controls (designated P1-P5) as well as in nontransfected PC12 cells, only a 45 kDa band was detected using a mouse monoclonal anti-synuclein antibody (FIG. 1). The 45 kDa band may represent endogenous post-translationally modified rat synuclein-1 or a cross-reacting protein (Stefanis et al., 2001, supra). Lysates of clonal lines transfected with wild-type α-synuclein (S4, S10, and S12) or mutant α-synuclein (M1, M13, M15, and M18) contained a broadband of 18 kDa that comigrated with the dominant band of α-synuclein detected in rat cortical lysates. Various levels of expression were achieved in different clonal lines. M1, M15, and M18, three lines expressing the highest levels of mutant α-synuclein, were comparable in terms of level of expression to S12, which expressed the wild-type form. Lines M13, S4, and S10 had considerably lower levels of expression. The levels of α-synuclein in the highest expressing lines were comparatively less than in rat cortex and therefore were well within the physiological range (FIG. 1). As reported, levels of the 18 kDa α-synuclein band are induced in PC12 cells with NGF treatment (Stefanis et al., 2001, supra). The levels of α-synuclein achieved in these lines were within the range of such induction.

B. Clonal Lines Expressing A53T α-synuclein Show Altered Morphological Characteristics.

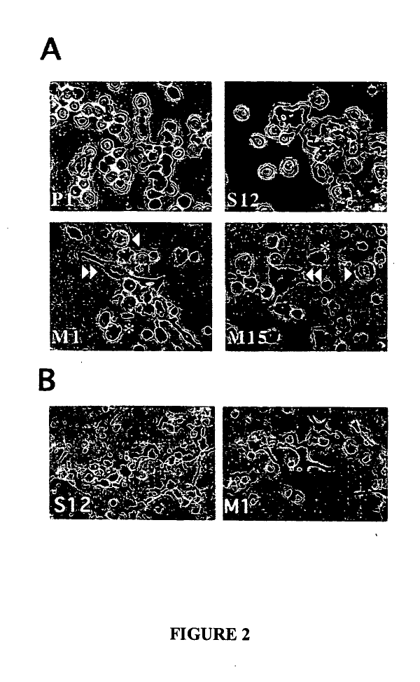

Cell lines expressing wild-type α-synuclein were indistinguishable from empty-vector control-transfected lines as well as nontransfected parental cells in terms of their appearance (FIG. 2) (Stefanis et al., 2001, supra). In contrast, M1 and M15 cells, which expressed high levels of mutant A53T α-synuclein, displayed a number of morphological abnormalities, including increased size, occasional stellate appearance, increased tendency to extend processes in the absence of NGF treatment, and increased cellular degeneration. Degenerating cells had a vacuolar-granular appearance (FIG. 2A). A similar phenotype was seen in the M18 line. Line M13, which expressed less of the exogenous mutant α-synuclein, displayed an intermediate appearance with no significant overall increase in cell size but the occasional presence of very large cells and some granular degenerating cells (data not shown).

PC12 cells treated with NGF assume a neuronal-like phenotype (Greene and Tischler, Establishment of a noradrenergic clonal line of rat adrenal pheochromocytoma cells which respond to nerve growth factor. Proc. Natl. Acad. Sci. USA, 73:2424-2428, 1976). Wild-type α-synuclein-expressing cells responded to treatment with 100 ng/ml NGF in a manner similar to that seen for empty-vector controls, establishing a neuritic network after 9 d of NGF treatment (FIG. 2B) (Stefanis et al., 2001, supra). In contrast, cells expressing mutant A53T α-synuclein showed a limited response to NGF, extending only short, stumpy processes. Many of these cells were very large and had bizarre shapes. As in the naive state, many granular-vacuolar degenerating cells were noted in these cultures (FIG. 2B).

C. Clonal Lines Expressing A53Tα-Synuclein Show Enhanced Cellular Degeneration.

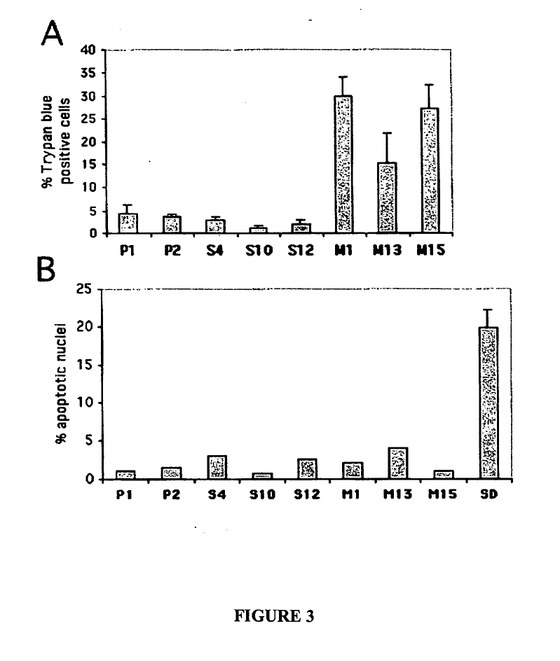

To quantify the impression of increased cellular degeneration in lines expressing mutant α-synuclein, the percentage of Trypan blue-positive cells in each line (FIG. 3A) was assessed. There was a markedly higher percentage of these degenerating cells in lines M1 and M15 compared with control cell lines or with lines expressing wild-type α-synuclein. Line M13 showed a more modest increase in degenerating cells (FIG. 3A).

To evaluate the morphological features of this cell death, nuclear dye Hoechst 33342 was used and the percentage of apoptotic nuclei was determined in each line. The proportion of apoptotic cells was low (<5%), and there was no significant difference in the relative percentage of apoptotic nuclei across cell lines. In contrast, a positive control of P1 cells undergoing death caused by serum deprivation showed a large proportion of apoptotic nuclei (FIG. 3B).

To test for potential involvement of caspases in cellular degeneration associated with expression of A53T α-synuclein, control, wild-type, and mutant lines were examined for basal caspase-3-like activity, as measured by cleavage of the fluorogenic substrate DEVD-AFC (15 μM; Enzyme Systems Products, Livermore, Calif.). No difference was found in basal activity among cell lines (data not shown). Positive controls from serum-deprived cultures showed the expected caspase-3-like activity (Stefanis et al., Induction of CPP32-like activity in PC12 cells by withdrawal of trophic support. J. Biol. Chem., 271:30663-30671, 1996). In addition, the pan-caspase inhibitor Boc-aspartate-fluoromethyl ketone (50 μM; Enzyme Systems Products, Livermore, Calif.) did not affect the percentage of Trypan blue-positive cells in the mutant lines (data not shown). It can be concluded that PC12 cell lines expressing A53T mutant α-synuclein display enhanced cellular degeneration that appears nonapoptotic by morphological and biochemical criteria.

D. Clonal Lines Expressing A53Tx-synuclein Show Cytoplasmic Ubiquitinated Aggregates.

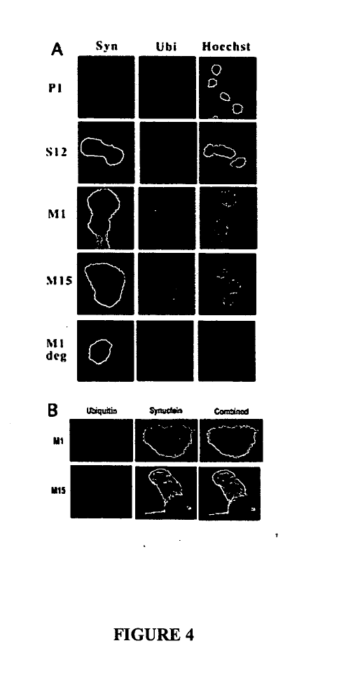

To evaluate further the nature of the different phenotypes in the lines expressing A53T α-synuclein, the various lines were immunostained with anti-synuclein. Empty-vector controls showed a low level of cytoplasmic staining (Stefanis et al., 2001, supra). A higher level of staining in the same distribution was seen in lines S4, S10, and M13 expressing low levels of α-synuclein. In contrast, lines S12, M1, and M15, which expressed higher α-synuclein levels, showed occasional nuclear staining in addition to cytoplasmic staining (FIGS. 4A, 4B). Overall, there was no detectable difference in the pattern of staining between lines expressing the wild-type or mutant α-synuclein.

The various cell lines were also stained with anti-ubiquitin in parallel. It was found that lines M1 and M15 showed a marked increase in ubiquitin staining compared with lines S12, S4, and S10 and empty-vector controls (FIG. 4A). Instead of the low-level diffuse staining seen in the other lines, the majority of M1 and M15 cells showed an intense punctate cytoplasmic staining. Cells of the M18 line showed a similar pattern, whereas M13 cells had an intermediate phenotype, with only occasional intense cytoplasmic ubiquitin staining (data not shown). To ensure that the increased level of ubiquitin staining was not simply a nonspecific response to stress, anti-ubiquitin antibody was also used to stain serum-deprived cells from the P1 clonal line. The intense cytoplasmic punctate staining was not seen under these circumstances (data not shown).

To characterize better the ubiquitin and synuclein immunostaining in the mutant lines, confocal microscopy was performed in these cell lines after staining them for ubiquitin and α-synuclein. Representative cells are shown from lines M1 and M15 (FIG. 4B). Synuclein staining was diffuse, occasionally including the nucleus. There was no evidence of synuclein aggregation. Immunostaining with a polyclonal synuclein antibody (Chemicon, Temecula, Calif.) also failed to show synuclein aggregation (data not shown). In contrast, ubiquitin immunostaining was primarily in the form of small discrete punctate accumulations of staining, which represent aggregates. Only a very limited proportion of these ubiquitinated aggregates stained for synuclein (FIG. 4B). Large ubiquitinated inclusions resembling Lewy bodies were not detected in these cells.

Cytoplasmic ubiquitinated aggregates were not seen in any of >10 wild-type α-synuclein-expressing lines, 5 empty-vector control lines, or multiple batches of untransfected PC12 cells. Furthermore, when some cultures derived from the two mutant lines, M1 and M115, eventually lost synuclein expression (when maintained in the absence of the selecting agent G418), they also lost the pattern of aggregated ubiquitin immunostaining (data not shown). This suggests that mutant α-synuclein expression is causally related to the accumulation of ubiquitinated aggregates.

In summary, PC12 cells expressing A53T α-synuclein show cytoplasmic ubiquitinated aggregates. These appear to be different from Lewy bodies in a number of respects, including their small size and the paucity of colocalization with α-synuclein.

E. A Number of Proteins, but Not α-synuclein Itself, are Preferentially Ubiquitinated in the Mutant α-synuclein Cell Lines.

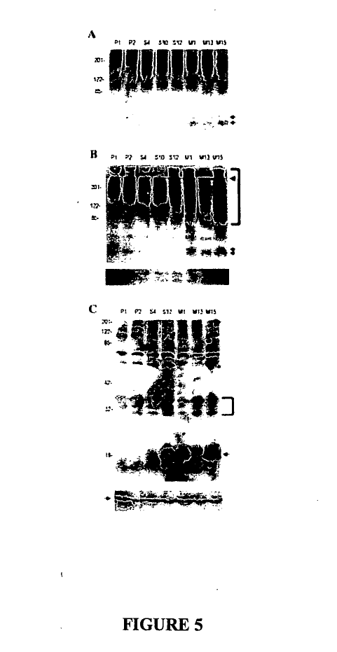

The antibody used for ubiquitin immunostaining could potentially identify free ubiquitin, mono-ubiquitinated, or polyubiquitinated proteins. To verify the immunostaining results and to assess whether the increased levels of ubiquitination in the mutant α-synuclein lines were attributable to polyubiquitinated proteins marked for degradation, Western immunoblotting was performed with the ubiquitin antibody. Cell lysates were divided into Triton-soluble and -insoluble fractions, and the latter was solubilized in SDS-sample buffer to assess the relative solubility of proteins. In Triton-soluble extracts there was little apparent difference in the high molecular mass pattern of ubiquitination among cell lines. A doublet at −52-54 kDa was more prominent in lines M1 and M15 compared with the other lines (FIG. 5A). The Triton-insoluble fractions of lines M15 and M1 showed a considerable increase in high molecular mass (>80 kDa) polyubiquitinated proteins, which are signaled for degradation, compared with controls or wild-type α-synuclein expressors (FIG. 5B, bracket). This increase was also apparent in the stacking gel, implying an increase of insoluble polyubiquitinated proteins. A few discrete bands, including bands migrating at 52-54 kDa, were more abundant in the mutant lines (FIG. 5B, arrows).

To test for the possibility that synuclein itself was ubiquitinated, Western blots of Triton-insoluble lysates from the various lines were probed with anti-synuclein. The characteristic ladder pattern of ubiquitination was not identified (FIG. 5C).

These results suggest that a number of proteins, but not α-synuclein itself, are preferentially polyubiquitinated in the mutant α-synuclein cell lines.

F. PC12 Cell Lines Expressing A53T α-synuclein Show Reduced Proteasomal Activity.

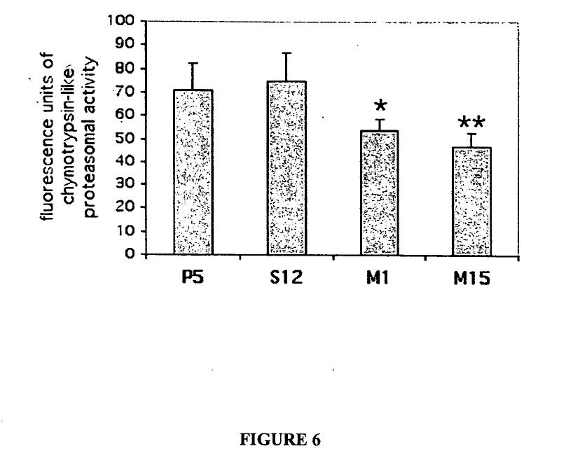

The phenotype of the lines expressing A53T α-synuclein is reminiscent in some respects of the phenotype of cells exposed to proteasomal inhibitors. These inhibitors can cause neurite sprouting, increased cell size, increased levels of polyubiquitinated proteins, and cell death (Drexler, Activation of the cell death program by inhibition of proteasome function. Proc. Natl. Acad. Sci. USA, 94:855-860, 1997; Lopes et al., p53-dependent induction of apoptosis by proteasome inhibitors. J. Biol. Chem., 272:12893-12896, 1997; Ohtani-Kaneko et al., Proteasome inhibitors which induce neurite outgrowth from PC12h cells cause different subcellular accumulations of multi-ubiquitin chains. Neurochem. Res., 23:1435-1443, 1998; Obin et al., Neurite outgrowth in PC12 cells: distinguishing the roles of ubiquitylation and ubiquitin-dependent proteolysis. J. Biol. Chem., 274:11789-11795, 1999; Rideout et al., Proteasomal inhibition leads to formation of ubiquitin/α-Synuclein-immunoreactive inclusions in PC12 cells. J. Neurochem., 78:899-908, 2001). To evaluate the possibility that the A53T cell lines had diminished proteasomal activity, the proteasomal chymotrypsin-like activity in various cell lysates was measured. M1 and M15 had 25-35% lower chymotrypsin-like activity compared with lines P5 or S12. This difference was statistically significant (FIG. 6).

G. PC12 Cell Lines Expressing A53T Mutant α-synuclein Show Accumulation of Vesicular Structures Suggestive of Autophagy.

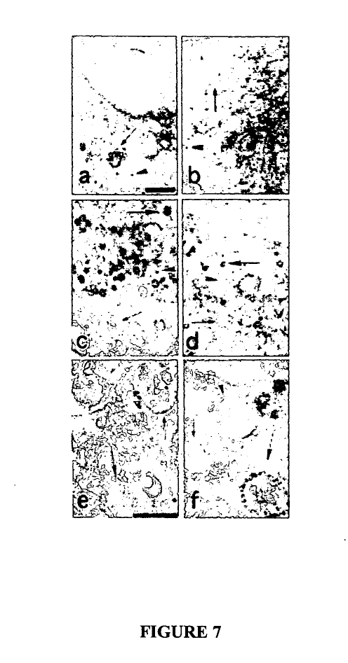

To further investigate the cellular degeneration seen in the A53T expressors, P1, S12, M1, and M15 cell lines were examined by electron microscopy. There was no evidence of increased apoptotic death in lines M1 or M15 compared with lines P1 and S12. There was however a marked accumulation of vesicular-autophagic structures in the A53T expressors. Many had double membranes. In many cases, these structures appeared to engulf intracellular organelles, in particular mitochondria. Some mitochondria were swollen or showed electron-dense material, consistent with calcification and degeneration. Other structures resembled more mature lysosomal organelles (FIG. 7). Another interesting feature of these lines was that there was a complete absence of dense core granules (DCGs), which are the catecholamine-secreting vesicular structures in PC12 cells. DCGs were readily apparent in the P1 and S12 lines (FIG. 7 and Table 1).

Together, these EM data suggest that the cellular degeneration seen in the A53T-expressing lines more closely resembled an autophagic form of cell death (Clarke, Developmental cell death: morphological diversity and multiple mechanisms. Anat. Embryol. (Berl), 181:195-213, 1990; Ohsawa et al., An ultrastructural and immunohistochemical study of PC12 cells during apoptosis induced by serum deprivation with special reference to autophagy and lysosomal cathepsins. Arch. Histol. Cytol., 61:395-403, 1998). In addition, these lines demonstrate an accumulation of aberrant vesicular structures that appear to be part of the lysosomal degradation system and show an absence of the dopamine-secreting dense core granules.

H. PC12 cell lines Expressing A53T Mutant α-synuclein do Not Release Dopamine After Depolarization.

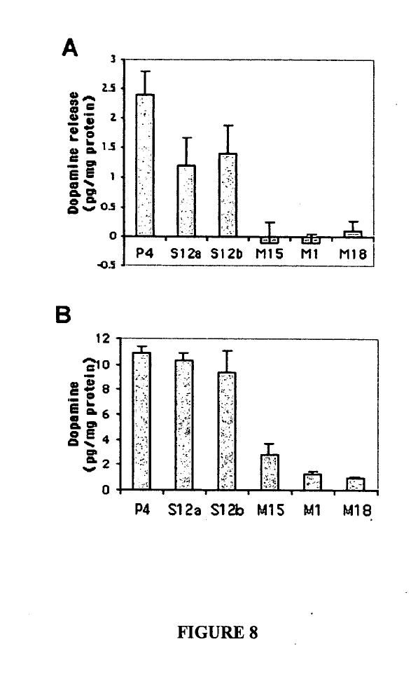

To investigate whether the absence of dense core granules in the A53T-expressing cells would have functional consequences, depolarization-induced dopamine release was measured from the various lines using HPLC. No stimulation-dependent dopamine release was observed from cultures of the M1, M15, or M18 lines (FIG. 8). In contrast, cultures expressing wild-type human α-synuclein retained the capacity for dopamine release, although to a somewhat reduced degree compared with an empty-vector control line (FIG. 8A). The mutant lines did contain intracellular dopamine, albeit at lower levels compared with the empty vector or the wild-type synuclein-expressing lines (FIG. 8B). Such lower intracellular levels could be attributable to degradation of nonsequestered dopamine or to mechanisms of feedback inhibition that operate when dopamine is prevented from entering the vesicular compartment (Goldstein and Greene, Activation of tyrosine hydroxylase by phosphorylation. In: Psychopharmacology: a third generation of progress (Meltzer H, ed), pp 75-80. New York: Raven, 1987; Fon et al., Vesicular transport regulates monoamine storage and release but is not essential for amphetamine action. Neuron, 19:1271-1283, 1997).

PC12 cells expressing mutant α-synuclein therefore manifest an absence of depolarization-induced dopamine release, consistent with the absence of dense core granules in these cells.

I. PC12 Cell Lines Expressing A53T Mutant α-synuclein Show Lysosomal Dysfunction.

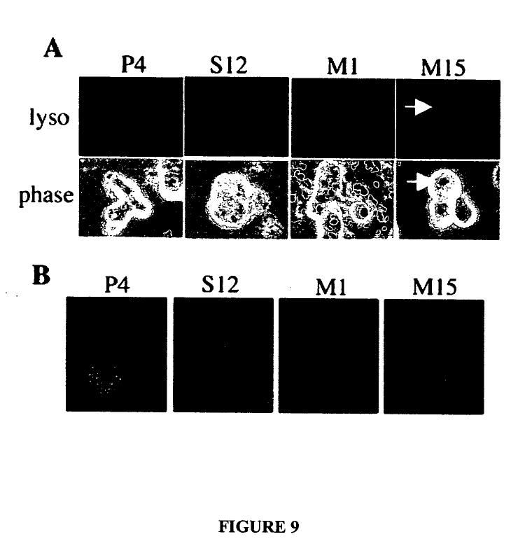

To examine whether the apparent increase in lysosomal-autophagic structures reflected an alteration in lysosomal activity in the A53T-expressing lines, the cells were labeled with an ionic dye, Lysotracker red (50 nM; Molecular Probes, Oregon), which is selectively sequestered in acidic organelles. In control cells and cells expressing wild-type α-synuclein, a fine punctate pattern of labeling was observed within the cytoplasm, consistent with lysosomal staining. There was a widespread, marked decrease of such punctate staining in cells from the M1 and M15 lines. Rare cells from the mutant lines (<2%) showed intense labeling of large cytosolic structures, which resembled vacuoles/inclusions (FIG. 9). A similar phenotype was seen in the M18 line (data not shown).

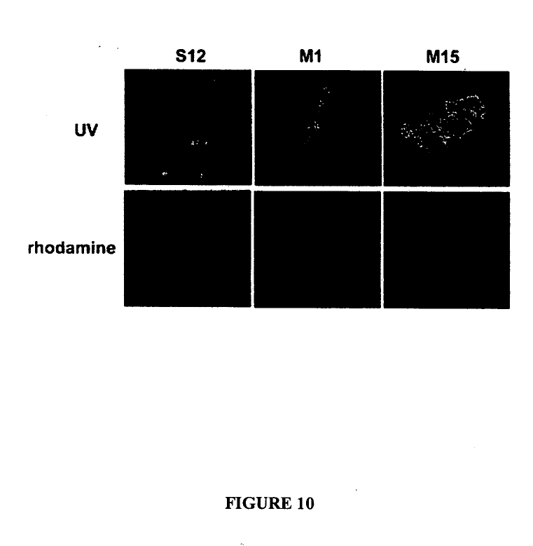

To further examine lysosomal function in these cell lines and to follow the fate of a lysosomal substrate within the cells, the cultures were incubated with Lysosensor Yellow/Blue Dextran, a substrate that is endocytosed by the cells and degraded in the lysosomes. The wavelength of the fluorescence emitted is pH-dependent. In an acidic environment, there is emission in the yellow spectrum; this was captured in experiments with a rhodamine filter. In a neutral or basic environment, emission is in the UV spectrum. After 20 hr incubation with Lysosensor Yellow/Blue Dextran, S12 cells showed intense punctate labeling in the rhodamine spectrum (red), indicative of normal lysosomal acidification, whereas M1 or M15 cells did not (FIG. 10, bottom row). All cell types showed punctate labeling with the UV filter (blue), but the A53T-expressing cells were of higher intensity (FIG. 10, top row). P4 cells did not show much fluorescence in either spectrum when incubated for 20 hr, presumably because the substrate had already been degraded; however, with 8 hr of incubation, P4 cells showed a pattern of labeling similar to the S12 cells (data not shown). The difference in incubation time needed to achieve similar levels of fluorescence may be related to different rates of endocytosis between the control and the synuclein-expressing lines. These results indicate that PC12 cells expressing mutant α-synuclein manifest an impairment of the lysosomal degradation system.

J. Limited Colocalization Between α-synuclein and Cathepsin D.

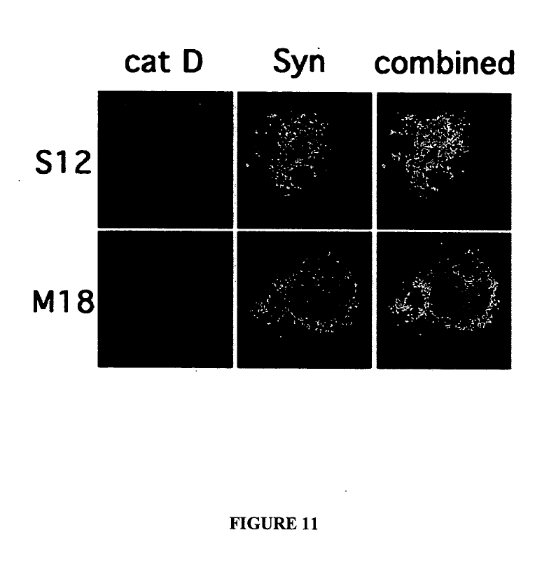

In view of the lysosomal dysfunction detected in the A53T mutant α-synuclein-expressing lines, it was desirable to test whether α-synuclein could directly interact with lysosomes to cause these effects. Cathepsin D is a major lysosomal enzyme, and immunostaining with antibodies directed against cathepsin D has been used as a lysosomal marker. Kegel et al. (Huntingtin expression stimulates endosomal-lysosomal activity, endosome tubulation, and autophagy. J. Neurosci., 20:7268-7278, 2000) recently reported that over expressed mutant Huntingtin colocalizes with cathepsin D in cytoplasmic vacuoles and stimulates autophagy in cloned striatal neuronal cells. Therefore, double immunostaining was performed for α-synuclein and cathepsin D in wild-type and mutant A53T-expressing cells and the cells were examined by confocal microscopy. Specific accumulation of α-synuclein was not found within cathepsin-positive structures. Overall, there was limited colocalization between α-synuclein and cathepsin D, and the extent of colocalization did not differ in cells expressing wild-type or mutant α-synuclein (FIG. 11).

The fact that α-synuclein does not significantly colocalize with cathepsin D suggests that the effects of mutant α-synuclein on lysosomal function may be mediated at an earlier stage of this degradation pathway, at the level of endosomes or early autophagosomes. Alternatively, the effects on lysosomal function may be indirect, caused by, for example, oxidative stress, which has been shown to induce lysosomal dysfunction (Brunk et al., Exposure of cells to nonlethal concentrations of hydrogen peroxide induces degeneration-repair mechanisms involving lysosomal destabilization. Free Radic. Biol. Med., 19:813-822, 1995).

### Example 12

Discussion

A. Expression of A53Tα-Synuclein Alters the Cellular Phenotype.

It has been demonstrated herein that expression of mutant A53T α-synuclein in PC12 cells leads to the formation of small ubiquitinated aggregates, to autophagic cellular degeneration, and to an absence of both DCGs and evoked dopamine release. M13, a line that had lower levels of the mutant protein, showed an intermediate phenotype, indicating that the effect is dose-dependent.

The levels of expression achieved were at least an order of magnitude lower compared with endogenous synuclein in rat brain on a per milligram of protein basis. At this expression level, there was a selective deleterious effect of mutant but not wild-type α-synuclein. Cell lines over expressing rat synuclein-1 were not generated, but, as reported previously, PC12 cells and sympathetic neurons upregulate levels of endogenous synuclein-1 in response to NGF, and this upregulation has no obvious detrimental effects (Stefanis et al., 2001, supra). Threonine at position 53 is the normal amino acid in rodents, and this has raised doubts about modeling the disease based on expression of this mutant form in rodent cells. However, human A53T α-synuclein is different from rodent synuclein-1 in a number of other amino acids. The inventors' results and the results of others (Zhou et al., Over expression of human α-synuclein causes dopamine neuron death in rat primary culture and immortalized mesencephalon-derived cells. Brain Res., 866:33-43, 2000) suggest that A53T mutant α-Synuclein, but not rat synculein-1, induces selective detrimental effects in a rodent background.

The inventors sought to replicate their findings in a different background of PC12 cells, by generating PC12 cell lines expressing the A53T or the A30P mutant or wild-type α-synuclein using a Tet-on system, with cells supplied by the manufacturer (Clontech, Palo Alto, Calif.). High levels of expression of α-synuclein were achieved in a number of lines, even without tetracycline, presumably because of the leakiness of the system. Wild-type α-Synuclein expressors were again indistinguishable from controls. Cells expressing the A53T or A30P mutant form did not demonstrate the dramatic phenotype seen in the original transfected lines, in that their size and morphological features appeared grossly normal and there was no enhancement of baseline cell death. However, these cells did show, albeit to a lesser degree than the original lines, small cytoplasmic ubiquitinated aggregates that again did not colocalize with α-synuclein (data not shown). The similarities and differences between the two sets of transfected cells underscore the potential importance of the cellular background or other unidentified factors in phenotype penetrance. Phenotypic heterogeneity has also been observed in families with α-synuclein mutations (Papadimitriou et al., Mutated α-synuclein gene in two Greek kindreds with familial PD: incomplete penetrance? Neurology, 52:651-654, 1999) and in transgenic mouse models over expressing wild-type or mutant α-synuclein (Masliah et al., Dopaminergic loss and inclusion body formation in α-synuclein mice: implications for neurodegenerative disorders. Science, 287:1265-1269, 2000; Matsuoka et al., Lack of nigral pathology in transgenic mice expressing human α-synuclein driven by the tyrosine hydroxylase promoter. Neurobiol. Dis., 8:535-539, 2001; Rathke-Hartlieb et al., Sensitivity to MPTP is not increased in Parkinson's disease-associated mutant-synuclein transgenic mice. J. Neurochem., 77:1181-1184, 2001). The inventors' cell lines described herein may thus present the opportunity to identify molecules that modulate the risk of degeneration associated with expression of mutant α-synuclein.

B. Effects of Mutant α-synuclein Appear Independent of Self-Aggregation: Potential Role of Proteasomal/Lysosomal Dysfunction.

Accumulating evidence suggests that α-synucleins, and in particular the mutant forms, aggregate in vitro (Conway et al., Accelerated in vitro fibril formation by a mutant α-synuclein linked to early-onset Parkinson disease. Nat. Med., 11:1318-1320, 1998; Giasson et al., Mutant and wild type human α-synucleins assemble into elongated filaments with distinct morphologies in vitro. J. Biol. Chem., 274:7619-7622, 1999), accumulate in Lewy bodies (Spillantini et al., α-Synuclein in Lewy bodies. Nature, 388:839-840, 1997; Spillantini et al., α-Synuclein in filamentous inclusions of Lewy bodies from Parkinson's disease and dementia with Lewy bodies. Proc. Natl. Acad. Sci. USA, 95:6469-6473, 1998) and, in an insoluble form, are increased in PD brains (Baba et al., Aggregation of α-synuclein in Lewy bodies of sporadic Parkinson's disease and dementia with Lewy bodies. Am. J. Pathol., 152:879-884, 1998), leading to the idea that α-synuclein forms aggregates that serve as a nidus for the formation of Lewy bodies, disruption of cell homeostasis, and death (Trojanowski et al., Fatal attractions: abnormal protein aggregation and neuron death in Parkinson's disease and Lewy body dementia. Cell Death Differ., 5:832-837, 1998). In the system of the present invention, there was no evidence of α-synuclein aggregation or ubiquitination or of significant colocalization of α-synuclein within the ubiquitinated aggregates. Therefore, it appears that A53T α-synuclein in the present system leads to the formation of ubiquitinated aggregates through an indirect mechanism. Similarly, α-synuclein expression enhances the formation of Huntingtin-containing inclusions but does not colocalize in the aggregates (Furlong et al., α-Synuclein over expression promotes aggregation of mutant huntingtin. Biochem. J, 15:577-581, 2000).

How could the ubiquitinated aggregates be formed then, if not by α-synuclein aggregation? The results herein indicate that the cells in the mutant lines have defects in the two major systems for the degradation of ubiquitinated proteins: lysosomes and the proteasome (Laszlo et al., Ubiquitinated protein conjugates are specifically enriched in the lysosomal system of fibroblasts. FEBS Lett., 261:365-368, 1990; Mayer et al., Ubiquitin, lysosomes, and neurodegenerative diseases. Ann. NY Acad. Sci., 674:149-160, 1992; Ciechanover, The ubiquitin-proteasome pathway: on protein death and cell life. EMBO J., 17:7151-7160, 1998). It is likely that such defects would lead to the accumulation of ubiquitinated aggregates: Inhibition of lysosomal cysteine proteases leads to the formation of ubiquitinated aggregates in cell culture and in vivo (Laszlo et al., 1990, supra; Cavanagh et al., Routes of excretion of neuronal lysosomal dense bodies after ventricular infusion of leupeptin in the rat: a study using ubiquitin and PGP 9.5 immunocytochemistry. J. Neurocytol., 22:779-791, 1993). Proteasomal inhibition leads to the accumulation of polyubiquitinated proteins (Ohtani-Kaneko et al., Proteasome inhibitors which induce neurite outgrowth from PC12h cells cause different subcellular accumulations of multi-ubiquitin chains. Neurochem. Res., 23:1435-1443, 1998). It should be noted however that the cellular phenotype observed after acute pharmacological proteasomal inhibition of PC12 cells (Rideout et al., Proteasomal inhibition leads to formation of ubiquitin/α-Synuclein-immunoreactive inclusions in PC12 cells. J. Neurochem., 78:899-908, 2001), although similar, differs in some respects from the one described here induced by mutant synuclein over expression. With acute proteasomal inhibition there is cell enlargement, neuritic extension, and induction of polyubiquitinated proteins and cytoplasmic ubiquitin immunostaining. However, one also detects apoptotic death and large single cytoplasmic inclusions that occasionally contain α-synuclein (Rideout et al., Proteasomal inhibition leads to formation of ubiquitin/α-Synuclein-immunoreactive inclusions in PC12 cells. J. Neurochem., 78:899-908, 2001). It is possible that more chronic, low-level regimens of pharmacological proteasomal inhibition would lead to a phenotype similar to the that shown by the cell lines of the present invention, or that additional factors, apart from proteasomal inhibition, may play a role in the generation of the mutant synuclein phenotype.

A recent study found inhibition of proteasomal activity in PC12 cells expressing A30P α-synuclein (Tanaka et al., Inducible expression of mutant α-synuclein decreases proteasome activity and increases sensitivity to mitochondria-dependent apoptosis. Hum. Mol. Genet., 10:919-926, 2001). The functional consequences of such inhibition were not examined. Nevertheless, these results, together with the present results, achieved with the other α-synuclein mutant (A53T), suggest a link between mutant α-synuclein expression and proteasomal inhibition. There is also evidence for increased sensitivity to proteasomal inhibition-induced death with expression of mutant or wild-type α-synuclein (Lee et al., Effect of the over expression of wild-type or mutant α-synuclein on cell susceptibility to insult. J. Neurochem., 76:998-1009, 2001; Tanaka et al., 2001, supra). A molecular basis for an interaction between α-synuclein and the proteasomal system is suggested by the association of α-synuclein with tat binding protein 1, a component of the proteasome (Ghee et al., Rat α-synuclein interacts with tat binding protein 1, a component of the 26S proteasomal complex. J. Neurochem., 75:2221-2224, 2000). Whether α-synuclein itself is degraded by the proteasome is controversial (Bennett et al., Degradation of α-synuclein by proteasome. J. Biol. Chem., 274:33855-33858, 1999; Ancolio et al., α-Synuclein and the Parkinson's disease-related mutant Ala53Thr-α-Synuclein do not undergo proteasomal degradation in HEK293 and neuronal cells. Neurosci. Lett., 285:79-82, 2000; Imai et al., Parkin suppresses unfolded protein stress-induced cell death through its E3 ubiquitin-protein ligase activity. J. Biol. Chem., 276:35661-35664, 2000; Rideout et al., Proteasomal inhibition leads to formation of ubiquitin/α-Synuclein-immunoreactive inclusions in PC12 cells. J. Neurochem., 78:899-908, 2001).

C. Death Associated With A53Tα-Synuclein Expression is Autophagic, Not Apoptotic.

The findings herein indicate that the increased cellular degeneration seen in the A53T lines is not attributable to apoptosis. Therefore, the present results diverge from those finding induction of apoptotic death with mutant α-synuclein over expression (Zhou et al., Over expression of human α-synuclein causes dopamine neuron death in rat primary culture and immortalized mesencephalon-derived cells. Brain Res., 866:33-43, 2000; Lee et al., Effect of the over expression of wild-type or mutant α-synuclein on cell susceptibility to insult. J. Neurochem., 76:998-1009, 2001). This may be related to the more chronic nature of mutant α-synuclein over expression in our model or to cell-specific differences. Induction of nonapoptotic death with mutant α-synuclein over expression has been reported previously, but the morphological features were not characterized further (Ostrerova et al., α-Synuclein shares physical and functional homology with 14-3-3 proteins. J. Neurosci., 19:5782-5791, 1999). Our EM data are suggestive of an autophagic mode of cell death (Clarke, Developmental cell death: morphological diversity and multiple mechanisms. Anat. Embryol. (Berl), 181:195-213, 1990; Ohsawa et al., An ultrastructural and immunohistochemical study of PC12 cells during apoptosis induced by serum deprivation with special reference to autophagy and lysosomal cathepsins. Arch. Histol. Cytol., 61:395-403, 1998) in which membrane-bound structures engulf intracellular organelles and participate in the destruction of the cell. Features of autophagy, but not death, were also reported in a neuronal cell line expressing murine α-synuclein (Hsu et al., α-Synuclein promotes mitochondrial deficit and oxidative stress. Am. J. Pathol., 157:401-410, 2000).

D. Effects on Catecholaminergic Activity.

One reason for using PC12 cells for the current studies was their dopaminergic phenotype. There is a striking deficiency of dense core granules (DCGs) in cell lines expressing A53T α-synuclein. Because DCGs are the major structures for storage and release of catecholamines (Sulzer and Pothos, Presynaptic mechanisms that regulate quantal size. Rev. Neurosci., 11:159-212, 2000), the mutant lines of the present invention were tested for evoked dopamine release, and it was found that this was totally absent. As noted below, such observations are novel and are potentially relevant to the dopaminergic deficits of PD.

E. Suggested Model of Cellular Dysfunction Induced by Mutant α-synuclein.

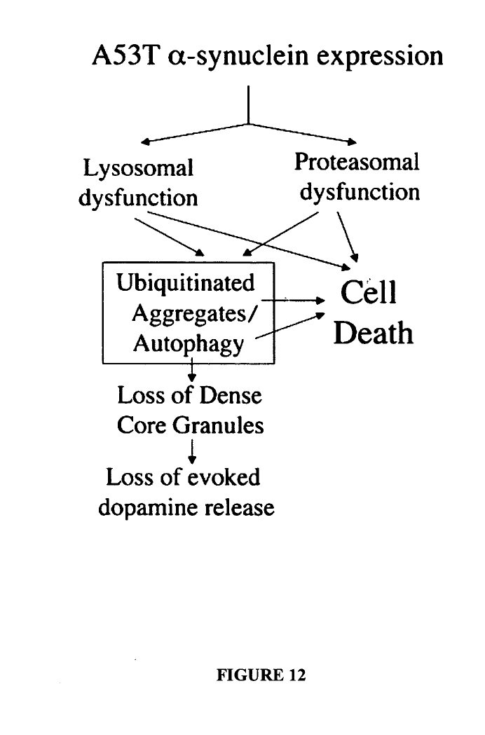

The multitude of abnormalities observed in the A53T α-synuclein-expressing cells makes it difficult to propose a causal sequence of events leading to cellular dysfunction and death. As mentioned above, it is likely that proteasomal and lysosomal dysfunction lead to ubiquitinated aggregate formation. Such dysfunction could also lead to autophagy, as described previously (Seglen et al., Structural aspects of autophagy. Adv. Exp. Med. Biol., 389:103-111, 1996; Wojcik et al., Ubiquitin-mediated proteolysis centers in HeLa cells: indication from studies of an inhibitor of the chymotrypsin-like activity of the proteasome. Eur. J. Cell. Biol., 71:311-318, 1996). It is tempting to speculate that the loss of DCGs and consequently of evoked dopamine release is attributable to DCG degradation within autophagic granules, as occurs with other intracellular organelles, such as mitochondria. Death could be attributable to protein aggregation or to the process of autophagy or could be directly related to lysosomal and proteasomal dysfunction or to a combination thereof (FIG. 12).

F. Insights and Potential Relevance to PD.

The findings described herein provide a number of novel insights into the function of mutant α-synuclein: the finding of the association of A30P α-synuclein with proteasomal dysfunction has been extended to the A53T mutant. Evidence of accumulation of polyubiquitinated proteins in the form of small cytoplasmic aggregates has also been provided. Further, the observation of lysosomal dysfunction as described herein has not been reported previously, and may play a role in protein aggregation and cellular, and particularly dopaminergic, dysfunction. This is also the first report of nonapoptotic, autophagic cell death induced by an α-synuclein mutant. This finding may have important implications, because the molecular pathways underlying these two different forms of death are distinct, although not mutually exclusive (Xue et al., Autophagy is activated by apoptotic signaling in sympathetic neurons: an alternative mechanism of death execution. Mol. Cell. Neurosci., 14:180-198, 1999; Bursch et al., Programmed cell death (PCD). Apoptosis, autophagic PCD, or others? Ann. NY Acad. Sci., 926:1-12, 2000). The issue of the relationship between α-synuclein aggregation and death is controversial. The present findings suggest that A53T α-synuclein can cause toxicity independently of its propensity to aggregate.

There are parallels between the present observations and PD. Specifically, the linking of mutant α-synuclein to the ubiquitin-dependent degradation system is especially important, in view of the genetic and pathological data linking defects in this system with PD (Kitada et al., Mutations in the parkin gene cause autosomal recessive juvenile parkinsonism. Nature, 392:605-608, 1998; Leroy et al., The ubiquitin pathway in Parkinson's disease. Nature, 395:451-452, 1998; Shimura et al., Familial Parkinson disease gene product, parkin, is a ubiquitin-protein ligase. Nat. Genet., 25:302-305, 2000; McNaught and Jenner, Proteasomal function is impaired in substantia nigra in Parkinson's disease. Neurosci. Lett., 297:191-194, 2001, McNaught et al., Failure of the ubiquitin-proteasome system in Parkinson's disease. Nat. Rev. Neurosci., 2:589-594, 2001). The dot-like cytoplasmic ubiquitinated aggregates are distinct from Lewy bodies but may be the nidus for the eventual formation of such larger inclusions in vivo. The issue of apoptosis in PD remains controversial (Burke, Apoptosis in degenerative diseases of the basal ganglia. The Neuroscientist, 4:301-311, 1998), whereas a recent EM study reported elements of autophagy in degenerating neurons in PD (Anglade et al., Apoptosis and autophagy in nigral neurons of patients with Parkinson's disease. Histol. Histopathol., 12:25-31, 1997). Notably, ultrastructural studies of Lewy bodies formed in sympathetic ganglion neurons of PD patients indicate that these bodies show more vesicular than filamentous features (Forno and Norville, Ultrastructure of Lewy bodies in the stellate ganglion. Acta. Neuropathol. (Berl), 34:183-197, 1976) and may share similarities with the vesicular-autophagic structures we have observed. Moreover, the novel effects observed on the dopaminergic release system may be related to the decreased availability of dopamine in nigrostriatal terminals out of proportion to the degree of nigral neuron loss in patients with PD (Hornykiewicz, Dopamine (3-hydroxytyramine) and brain function. Pharmacol. Rev., 18:925-965, 1996). Mutant α-Synuclein-expressing PC12 cells may serve as a useful model to study the molecular mechanisms through which aberrant α-synuclein leads to dopaminergic neuron dysfunction and degeneration in PD.

TABLE 1
Absence of DCGs in PC12 cell lines
expressing mutant α-synuclein

| | Mean number of | |
|---|---|---|
| | DCGs per section | SEM |
| P1 | 54.9 | 21.1 |
| S12 | 113.8 | 34 |
| M1 | 0.8 | 0.6 |
| M15 | 1 | 0.7 |

PC12 cells from the various lines were fixed and processed for electron microscopy. For each line, nine random sections of 10 × 13 μm were scanned and the number of DCGs was assessed.

All publications, patent applications and issued patents cited in this specification are herein incorporated by reference as if each individual publication, patent application or issued patent were specifically and individually indicated to be incorporated by reference. Further, the earlier incorporation by reference of any specific publication, patent application or issued patent shall not negate this paragraph. The citation of any publication, patent application or issued patent is for its disclosure prior to the filing date of the subject application and should not be construed as an admission that the present invention is not entitled to antedate such disclosure by virtue of prior invention.

We claim:

1. A method for generating a dopaminergic cell line stably expressing human A53T α-synuclein, the method comprising the steps of:
(a) introducing an expression vector into a population of rat pheochromocytoma PC12 cells, wherein the expression vector comprises a sequence encoding human A53T α-synuclein operatively linked to and under the control of a promoter; and
(b) isolating PC12 cell lines stably expressing human A53T α-synuclein.

2. The method of claim 1, wherein the expression vector further comprises a sequence encoding a selectable marker gene.

3. The method of claim 2, wherein the selectable marker gene is one of neo, hyg, pac, zeo or gpt.

4. The method of claim 3, wherein the selectable marker gene is neo.

5. The method of claim 2, wherein the PC12 cells stably expressing human A53T α-synuclein are isolated by virtue of growth on a selective medium, wherein growth on the selective medium indicates successful introduction and expression of the expression vector.

6. The method of claim 5, wherein the selective marker gene is neo and the selective medium comprises neomycin.

7. The method of claim 2, wherein the promoter is an inducible promoter.

8. The method of claim 7, wherein the inducible promoter is selected from the group consisting of a tetracycline-responsive promoter, an ecdysone-inducible promoter, a metallothionein-regulated promoter, a steroid-regulated promoter, and a heat-shock regulated promoter.

9. The method of claim 1, wherein the expression vector is a plasmid vector, a phasmid vector, an adenoviral vector, an adenoassociated vector, a vaccinia viral vector, a lentiviral vector, a herpes viral vector or a retroviral vector.

10. The method of claim 9, wherein the expression vector is a plasmid vector.

11. The method of claim 10, wherein the plasmid vector is pcDNA3.

12. The method of claim 2, wherein the expression vector is introduced via calcium phosphate transfection, liposome-mediated transfection, microprojectile-mediated delivery, electroporation, biolistic transfection, microinjection, or viral-mediated transfection.

13. The method of claim 12, wherein the expression vector is introduced via electroporation.

14. The method of claim 1, wherein a cell of the isolated cell line exhibits proteasomal dysfunction as compared to a cell from a suitable control cell line not expressing mutant α-synuclein.

15. The method of claim 14, wherein proteasomal dysfunction is determined by the increased presence of ubiquinated aggregates in the cytoplasm of a cell of the isolated cell line, as compared to a cell from a suitable control cell line not expressing mutant α-synuclein.

16. The method of claim 14, wherein proteasomal dysfunction is determined by a significantly decreased proteasomal chymotrypsin-like activity in a cell of the cell line, as compared to a cell from a suitable control cell line not expressing mutant α-synuclein.

17. The method of claim 1, wherein a cell of the isolated cell line exhibits dopaminergic dysfunction, as compared to a cell from a suitable control cell line not expressing mutant α-synuclein.

18. The method of claim 17, wherein dopaminergic dysfunction is determined by an absence of dense core granules in a cell of the cell line.

19. The method of claim 17, wherein dopaminergic dysfunction is determined by an absence of evoked dopamine release by a cell of the cell line.

20. The method of claim 1, wherein a cell of the isolated cell line exhibits lysosomal dysfunction, as compared to a cell from a suitable control cell line not expressing mutant α-synuclein.

21. The method of claim 20, wherein lysosomal dysfunction is determined by the accumulation of lysosomal-autophagic structures in a cell of the cell line.

22. The method of claim 1, wherein cells of the isolated cell line exhibit increased non-apoptotic cell death, as compared to a cell from a suitable control cell line not expressing mutant α-synuclein.

23. The method of claim 1, wherein a cell of the isolated cell line exhibits (i) proteasomal dysfunction, (ii) dopaminergic dysfunction, (iii) lysosomal dysfunction and (iv) increased non-apoptotic cell death, as compared to a cell from a suitable control cell line not expressing mutant α-synuclein.

24. A PC12 cell line stably expressing human A53T α-synuclein, wherein a cell of the cell line is characterized by: (i) proteasomal dysfunction; (ii) dopaminergic dysfunction; (iii) lysosomal dysfunction; and (iv) increased non-apoptotic cell death, as compared to a cell from a suitable control cell line not expressing mutant α-synuclein.

25. The PC12 cell line of claim 23, wherein a cell of the cell line is further characterized by: (i) the presence of ubiquinated aggregates in the cytoplasm; (ii) significantly decreased proteasomal chymotrypsin-like activity; (iii) an absence of dense core granules; (iv) an absence of evoked dopamine release; and (v) an accumulation of lysosomal-autophagic structures.

26. A method for producing a cellular model of Parkinson's disease comprising the steps of:
(a) introducing an expression vector into a population of rat pheochromocytoma PC12 cells, wherein the expression vector comprises a sequence encoding human A53T α-synuclein operatively linked to and under the control of a promoter; and
(b) isolating PC12 cell lines stably expressing human A53T α-synuclein, wherein the cells of the isolated PC12 cell lines are characterized by (i) proteasomal dysfunction; (ii) dopaminergic dysfunction; (iii) lysosomal dysfunction; and (iv) increased non-apoptotic cell death.

27. The method of claim 26, wherein the cells of the isolated PC12 cell lines are further characterized by: (i) the presence of ubiquinated aggregates in the cytoplasm; (ii) significantly decreased proteasomal chymotrypsin-like activity; (iii) an absence of dense core granules; (iv) an absence of evoked dopamine release; and (v) an accumulation of lysosomal-autophagic structures.

28. A method of identifying an agent that inhibits cellular degeneration associated with the expression of mutant α-synuclein, the method comprising the steps of:
(a) obtaining a PC12 cell line stably expressing human A53T α-synuclein, wherein cells of the cell line are characterized by increased non-apoptotic cell death;
(b) contacting the cells of the PC12 cell line with an agent of interest;
(c) comparing the phenotype of the contacted cells with the phenotype of cells from a suitable control line; and
determining the effect of the agent of interest on the contacted cells, wherein a phenotype of the contacted cells associated with reduced cellular degeneration indicates that the agent of interest has an inhibitory effect on cellular degeneration associated with the expression of mutant α-synuclein.

29. The method of claim 28, wherein a reduction of non-apoptotic cell death in the contacted cells as compared to cells of a suitable control line indicates that the agent has an inhibitory effect on cellular degeneration associated with the expression of mutant α-Synuclein.

30. The method of claim 28, where the cells of the cell line stably expressing human A53T α-synuclein are further characterized by: (i) proteasomal dysfunction; (ii) dopaminergic dysfunction; and (iii) lysosomal dysfunction.

31. The agent identified by the method of claim 30.

32. A method of identifying an agent that inhibits dopaminergic dysfunction associated with the expression of mutant α-synuclein, the method comprising the steps of:
(a) obtaining a PC12 cell line stably expressing human A53T α-synuclein, wherein cells of the cell line are characterized by dopaminergic dysfunction;
(b) contacting the cells of the PC12 cell line with an agent of interest;
(c) comparing the phenotype of the contacted cells with the phenotype of cells from a suitable control line; and
(d) determining the effect of the agent of interest on the contacted cells, wherein a phenotype of the contacted cells associated with increased dopaminergic function indicates that the agent of interest has an inhibitory effect on dopaminergic dysfunction associated with the expression of mutant α-synuclein.

33. The method of claim 32, wherein an increase of intracellular dopamine levels in the contacted cells as compared to cells of a suitable control line indicates that the agent of interest has an inhibitory effect on dopaminergic dysfunction associated with the expression of mutant α-synuclein.

34. The agent identified by the method of claim 33.

35. The method of claim 32, wherein the ability of a contacted cell to undergo evoked dopamine release indicates that the agent of interest has an inhibitory effect on dopaminergic dysfunction associated with the expression of mutant α-synuclein.

36. The agent identified by the method of claim 35.

37. The method of claim 32, wherein the presence of dense core granules in the cytoplasm of the contacted cells indicates that the agent of interest has an inhibitory effect on dopaminergic dysfunction associated with the expression of mutant α-synuclein.

38. The agent identified by the method of claim 37.

39. The method of claim 32, wherein the cells of the cell line stably expressing human A53T α-synuclein are further characterized by: (i) proteasomal dysfunction; (ii) lysosomal dysfunction, and (iii) increased non-apoptotic cell death.

40. A method of identifying an agent that inhibits proteasomal dysfunction associated with the expression of mutant α-synuclein, the method comprising the steps of:
(a) obtaining a PC12 cell line stably expressing human A53T α-synuclein, wherein cells of the cell line are characterized by proteasomal dysfunction;
(b) contacting the cells of the PC12 cell line with an agent of interest;
(c) comparing the phenotype of the contacted cells with the phenotype of cells from a suitable control line; and
(d) determining the effect of the agent of interest on the contacted cells,
wherein a phenotype of the contacted cells associated with increased proteasomal activity indicates that the agent of interest has an inhibitory effect on proteasomal dysfunction associated with the expression of mutant α-synuclein.

41. The method of claim 40, wherein an increased proteasomal chymotrypsin-like activity in the contacted cells as compared to the cells of a suitable control line indicate that the agent has an inhibitory effect on proteasomal dysfunction associated with the expression of mutant α-synuclein.

42. The agent identified by the method of claim 39.

43. The method of claim 40, wherein a decrease in the presence of ubiquinated aggregates in the contacted cells as compared to the cells of a suitable control line indicate that the agent has an inhibitory effect on proteasomal dysfunction associated with the expression of mutant α-synuclein.

44. The agent identified by the method of claim 43.

45. The method of claim 40, wherein the cells of the cell line stably expressing human A53T α-synuclein are further characterized by: (i) dopaminergic dysfunction; (ii) lysosomal dysfunction; and (iii) increased non-apoptotic cell death.

46. A method of identifying an agent that inhibits lysosomal dysfunction associated with the expression of mutant alpha-synuclein, the method comprising the steps of:
(a) obtaining a PC12 cell line stably expressing human A53T α-synuclein, wherein cells of the cell line are characterized by lysosomal dysfunction;
(b) contacting the cells of the PC12 cell line with an agent of interest;
(c) comparing the phenotype of the contacted cells with the phenotype of cells from a suitable control line; and
(d) determining the effect of the agent of interest on the contacted cells,
wherein a phenotype of the contacted cells associated with increased lysosomal activity indicates that the agent of interest has an inhibitory effect on lysosomal dysfunction associated with the expression of mutant α-synuclein.

47. The method of claim 46, wherein a decrease of the presence of lysosomal-autophagic structures in the cytoplasm of the contacted cells as compared to the cells of a suitable control line indicates that the agent has an inhibitory effect on lysosomal dysfunction associated with the expression of mutant α-synuclein.

48. The agent identified by the method of claim 47.

49. The method of claim 46, wherein an increase of fine, punctate staining with an ionic dye in the contacted cells as compared to the cells of a suitable control line indicate that the agent has an inhibitory effect on lysosomal dysfunction associated with the expression of mutant α-synuclein.

50. The agent identified by the method of claim 49.

51. The method of claim 46, wherein an increase of acidification and degradation of a lysosomal substrate in the contacted cells as compared to the cells of a suitable control line indicate that the agent has an inhibitory effect on lysosomal dysfunction associated with the expression of mutant α-synuclein.

52. The agent identified by the method of claim 51.

53. The method of claim 46, wherein the cells of the cell line stably expressing human A53T α-synuclein are further characterized by: (i) proteasomal dysfunction; (ii) dopaminergic dysfunction; and (iii) increased non-apoptotic cell death.

54. A method of screening an agent to determine its potential effectiveness in the treatment of a synucleinopathic neurodegenerative disorder, the method comprising the steps of:
(a) obtaining a PC12 cell line stably expressing human A53T α-synuclein, wherein cells of the cell line are characterized by: (i) proteasomal dysfunction; (ii) dopaminergic dysfunction; (iii) lysosomal dysfunction; and (iv) increased non-apoptotic cell death;
(b) contacting the cells of the PC12 cell line with an agent of interest;
(c) comparing the phenotype of the contacted cells with the phenotype of cells from a suitable control line; and
(d) determining the effect of the agent of interest on the contacted cells,
wherein a phenotype of the contacted cells associated with (i) decreased proteasomal dysfunction; (ii) decreased dopaminergic dysfunction; (iii) decreased lysosomal dysfunction; or (iv) decreased non-apoptotic cell death, when compared to the cells of a suitable control line, indicates that the agent of interest is potentially effective in treating a synucleinopathic neurodegenerative disorder.

55. The method of claim 54, wherein the synucleinopathic neurodegenerative disorder is one of Parkinson's Disease, Dementia with Lewy Bodies, Lewy Body Variant of Alzheimer's Disease, Multiple System Atrophy and Hallervorden-Spatz syndrome.

56. The method of claim 55, wherein the Multiple System Atrophy is one of Shy-Drager Syndrome, striatonigral degeneration, and olivopontocerebellar atrophy.

57. The method of claim 55, wherein the disease is Parkinson's Disease.

58. The agent identified by the method of claim 57.
